(19) United States
(12) Patent Application Publication
(10) Pub. No.: US 2026/0199418 A1
(43) Pub. Date: Jul. 16, 2026

(54) ANTI-FERTILITY AND ANTI-HUMAN CHORIONIC GONADOTROPIN (HCG) ACTIVITY PLANT SUBSTANCES IN THE TREATMENT OF CANCER

(71) Applicant: Andrew Lange, Sausalito, CA (US)

(72) Inventor: Andrew Lange, Sausalito, CA (US)

(21) Appl. No.: 18/790,186

(22) Filed: Jan. 2, 2025

(51) Int. Cl. A61K 36/58; A61K 36/185; A61K 36/42; A61K 36/428; A61K 36/539; A61K 38/16; A61K 45/06; A61P 35/00

(52) U.S. Cl. CPC A61K 36/58; A61K 36/42; A61K 36/428; A61K 36/539; A61K 36/577; A61K 36/5775; A61K 38/168; A61K 45/06; A61P 35/00; A61K 2236/39

(57) ABSTRACT

The present description broadly relates to compounds that have anti-fertility effects or the ability to inhibit human chorionic gonadotropin (hCG) in biological systems. This includes compounds both synthetic and non-synthetic, whose structure are derived from those of plants and other natural substances that have the ability to inhibit human chorionic gonadotropin in biological systems. Examples of these plants are, but are not limited to Azadirachta indica (neem), Gossypium herbaceum, Trichosanthes kirilowii, Hibiscus rosa-sinensis (Malvaceae), Momordica charantia and Scutellaria barbata. In some embodiments, the compounds can be used for the treatment of cancer and tumors which have biomarkers for chorionic antigens. In addition to the anti-hCG activities of the plant substances there may be additional or corollary actions which may contribute to their efficacy.

### BACKGROUND

### Field

The present disclosure generally relates to compositions, formulations and methods of treating cancer and boosting immune system using plant substances. The plant substances are compounds with anti-hCG activity and/or anti-fertility activity.

### Description of Related Art

Human Chorionic Gonadotropin or hCG is found in cells throughout the body. It is found in sperm and egg. It is produced in small quantities in pulsations by the pituitary gland in the brain. Its highly negatively charged surface makes it immunologically inert. Normal immune system cells from the body cannot destroy it.

The placenta richly produces Human Chorionic Gonadotropin, or hCG during pregnancy. The placenta is the tissue or organ formed during gestation through which the mother's blood and nutrition is transferred to the fetus. It is the barrier that protects the fetus from infections present in the mother.

The family of glycoproteins, to which hCG belongs, is not a recent evolutionary development. Similar proteins have been found in mammals and insects. hCG-like proteins have been isolated in bacteria and yeasts such as Candida albicans.

Normally foreign chromosomes are rejected by the body's defenses. Even as a single cell, the union of the parent's chromosomes could be considered as a foreign invading body, much like any foreign bacteria or virus. hCG veils the newly implanted embryo from being rejected as foreign by the mother's body. It is therefore known as the hormone of pregnancy. hCG also protects the aggressive spermatozoon as they make their journey towards the egg, so that the female's immune system will not be able to reject the sperm as foreign invaders.

hCG can be found in tumor and cancer cells. It is not believed to be the cause of cancer, but it is activated when malignant transformation occurs. hCG may protect cancer cell from being recognized or attacked by the immune system. This is why tumor formation is so insidiously proliferated, undetected until the growth is so great that it interrupts function in some neighboring structure. By the time cancer is detected clinically it has usually been forming for some time. The presence of hCG also masks the metastatic dissemination of cancer cells.

In 1902 the Scottish embryologist John Beard noticed the relationship between the growth of the trophoblastic cells that surround a fertilized egg and cancer cells. These trophoblasts attach to the uterus forming the chorionic villi that are the fetus's portion of the placenta. The rapid division, growth and invasive action of the trophoblasts and placenta were recognized as being correlates for the behavior of cancer. In fact current methods of treating cancer by stopping the process of angiogenesis (growth of the tumor into surrounding tissue) originate in Beard's discovery. Unfortunately this method does not address the metastatic potential of cancer.

In 1972, Valentin I. Govallo M. D., PhD, Director of the Laboratory of Immunology in Moscow, published The Immunology of Pregnancy and Cancer. In his book, he describes his use of an extract of the human chorionic villi from the placenta combined with white blood cells to immunize the patient against the placenta-like cancer. He compared this with other methods of immunotherapy in which he specifically attempted to induce immunoglobulin and natural killer cell responses. The use of placental extracts, that he calls immunoembryotherapy, has had remarkable success.

Govallo reported that out of 35 patients treated, 88.6% survived for up to three years, 77.1% survived for five years and 65.4% survived for 10 years or more. This was in a wide variety of cancers including breast, lung, uterine and kidney.

### SUMMARY

In some embodiments, a method of treating cancer is provided. The method comprises administering a compound to a subject in need thereof. The compound is an anti-hCG compound. The compound is an anti-hCG compound in one or more of Azadirachta indica (neem), Gossypium herbaceum, Trichosanthes kirilowii, Hibiscus rosa-sinensis (Malvaceae) or Scutellaria barbata, and the compound is administered in an amount that is adequate to treat cancer.

In some embodiments, a method of treating cancer is provided. The method comprises administering an herbal substance to boost immune system in a subject. The herbal substance comprises an anti-hCG compound in one or more of Azadirachta indica (neem), Gossypium herbaceum, Trichosanthes kirilowii, Hibiscus rosa-sinensis (Malvaceae) or Scutellaria barbata. The anti-hCG compound is administered in an amount that is adequate to treat cancer.

In some embodiments, a composition for treating cancer is provided. The composition is an herbal substance to boost immune system in a subject. The herbal substance comprises an anti-hCG compound in one or more of Azadirachta indica (neem), Gossypium herbaceum, Trichosanthes kirilowii, Hibiscus rosa-sinensis (Malvaceae) or Scutellaria barbata. The anti-hCG compound is administered in an amount that is adequate to treat cancer.

### BRIEF DESCRIPTION OF THE DRAWINGS

FIG. 1 depicts a graph of the level of cancer antigens (CEA AND CA2729) detected over time in a 40 year old female subject suffering from metastatic breast cancer with multiple bone tumors.

FIGS. 2A-B are graphical illustrations depicting comparative plots of cell lines according to different treatments.

FIG. 3 is a graphical illustration depicting comparative results of taxol use alone and in combination with experimental drugs dissolved in ethanol.

FIGS. 4A-B are graphical illustrations depicting comparative cell line results according to different treatments.

FIG. 5 is a graphical illustration depicting comparative results of drug treatments at various concentrations.

FIG. 6 is a graphical illustration depicting comparative effects of gemcitabine on cell lines.

FIG. 7 is a graphical illustration depicting comparative effects of experimental drugs in higher concentrations.

FIG. 8 is a graphical illustration depicting comparative effects of cell viability according to different treatments.

FIG. 9 is a graphical illustration depicting comparative results of experimental drug treatments with respect to miR-21 expression.

FIG. 10 is a graphical illustration depicting comparative results of experimental drug treatments with respect to ECAD and VIM expression.

### DETAILED DESCRIPTION

One of the greatest problems in the treatment of cancer is the inability of the body's immune system to recognize and eradicate cancer cells. Thus, novel treatment options that either treat cancer directly or boost immune system or both are required.

Human Chorionic Gonadotropin or hCG protects the cancer cell from being recognized or attacked by the immune system. It is in a true sense a cloaking device for cancer cells, which allows them to proliferate and metastasize. A positive marker for chorionic antigens shows the presence of hCG in the cancer cells. These antigens are used clinically to track the presence and progress of cancers and the efficacy of treatment.

Certain plants and plant compounds are known to have anti-fertility or inhibitory effects on hCG. Thus, an approach that utilizes plants and more specifically their purified, isolated, and/or concentrated compounds or synthetic analogues, that have been shown to have 1) anti-fertility effects, 2) have inhibitory effects on hCG, or 3) both, in the treatment of cancers in which there is a positive marker for a chorionic antigen can be very useful.

### Definitions

The terms “cancer”, “neoplasia” and “neoplastic disease” are used interchangeably and refer to a cell or population of cells whose growth, proliferation or survival is greater than the growth, proliferation or survival of a normal counterpart cell, e.g. a cell proliferative or differentiative disorders. Typically, the growth is uncontrolled. The term “malignancy” refers to invasion of nearby tissue. The term “metastasis” refers to spread or dissemination of a cancer or neoplasia to other sites, locations or regions within the subject, in which the sites, locations or regions are distinct from the primary cancer. In some embodiments, a cancer is malignant and presents with elevated hCG markers. For example, the level of hCG can be higher in a malignant tumor than in healthy, noncancerous tissue.

The term “tumor” denotes a benign growth, and need not present with elevated hCG markers.

As used in this specification and the appended claims, the singular forms “a,” “an” and “the” include plural references unless the content clearly dictates otherwise.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

In some embodiments, the “purity” of any given agent (that has, for example, been purified from a plant as described herein) in a composition may be specifically defined. For instance, certain compositions may comprise an agent that is at least 80, 85, 90, 91, 92, 93, 94, 95, 96, 97, 98, 99, or 100% pure, including all decimals and ranges in between, as measured, for example and by no means limiting, by high performance liquid chromatography (HPLC), a well-known form of column chromatography used frequently in biochemistry and analytical chemistry to separate, identify, and quantify compounds.

As used herein, “effective treatment” refers to treatment producing a beneficial effect, e.g., amelioration of at least one symptom of a disease or disorder. A beneficial effect can take the form of an improvement over baseline, e.g, an improvement over a measurement or observation made prior to initiation of therapy according to the method. A beneficial effect can also take the form of arresting, slowing, retarding, or stabilizing of a deleterious progression of a marker of a cancer. Effective treatment may refer to alleviation of at least one symptom of a cancer. Such effective treatment can, e.g., reduce patient pain, reduce the size and/or number of lesions, may reduce or prevent metastasis of a cancer tumor, and/or may slow growth of a cancer tumor.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to cancers, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay tumor development. In some embodiments, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit (i.e., slow to some extent and may stop) tumor metastasis; (v) inhibit tumor growth; (vi) prevent or delay occurrence and/or recurrence of tumor; and/or (vii) relieve to some extent one or more of the symptoms associated with the cancer.

The terms “combination therapy,” “co-administration,” “co-administered” or “concurrent administration” (or minor variations of these terms) include simultaneous administration of at least two therapeutic agents to a patient or their sequential administration within a time period during which the first administered therapeutic agent is still present in the patient when the second administered therapeutic agent is administered.

The term “monotherapy” refers to administering a single drug to treat a disease or disorder in the absence of co-administration of any other therapeutic agent that is being administered to treat the same disease or disorder.

“Dosage” refers to parameters for administering a drug in defined quantities per unit time (e.g., per hour, per day, per week, per month, etc.) to a patient. Such parameters include, e.g., the size of each dose. Such parameters also include the configuration of each dose, which may be administered as one or more units, e.g., taken at a single administration, e.g., orally (e.g., as one, two, three or more pills, capsules, etc.) or injected (e.g., as a bolus). Dosage sizes may also relate to doses that are administered continuously (e.g., as an intravenous infusion over a period of minutes or hours). Such parameters further include frequency of administration of separate doses, which frequency may change over time.

“Dose” refers to an amount of a drug given in a single administration.

In some embodiments, the present disclosure relates to methods and compositions for the prevention and/or treatment of tumor and/or cancer. Any of the methods provided herein can be combined with any of the compositions provided herein and vice versa.

In some embodiments, the present disclosure relates to methods and compositions for the prevention and/or treatment of tumor and/or cancer using plant substances and their derivatives both natural and synthetic with anti-fertility and/or anti-human chorionic gonadotropin activities.

In some embodiments, the present application provides methods and compositions for the prevention and/or treatment of cancers based on the active compounds in these plant substances. Some embodiments of the present disclosure provide methods and compositions for the prevention and/or treatment of cancers, tumors or both that have biomarkers for chorionic antigens.

Some embodiments of the methods and compositions utilize compounds that are derived from the plants including, but are not limited to, Azadirachta indica (neem), Gossypium herbaceum, Trichosanthes kirilowii, Hibiscus rosa-sinensis (Malvaceae), and/or Scutellaria barbata.

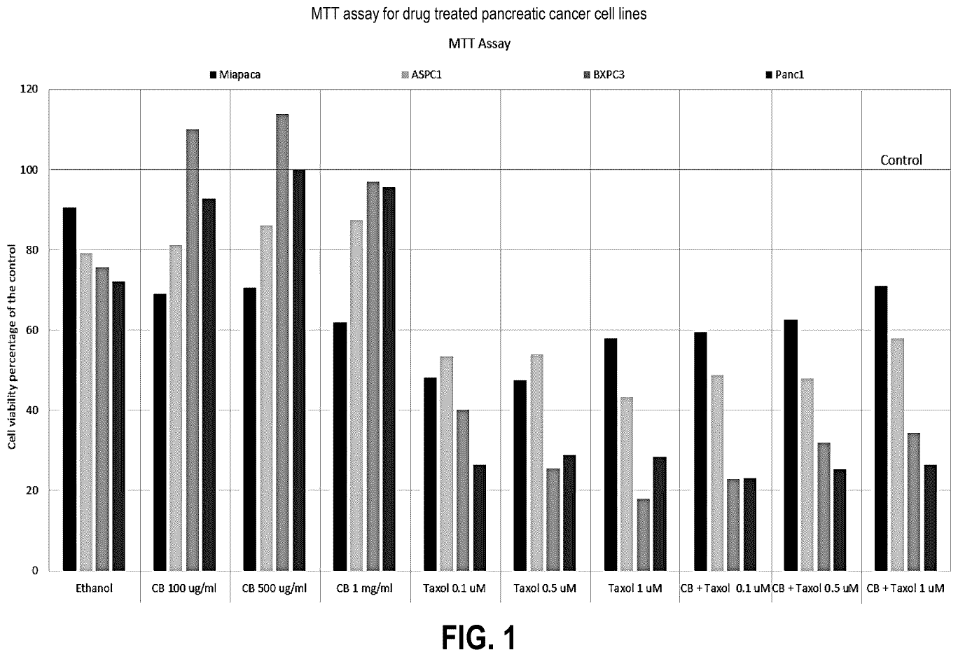

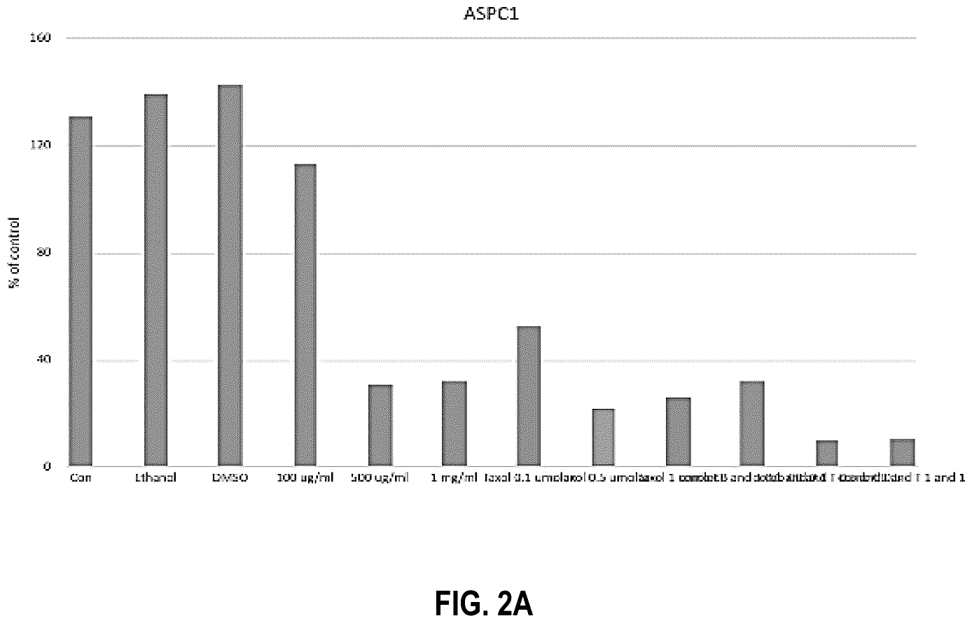

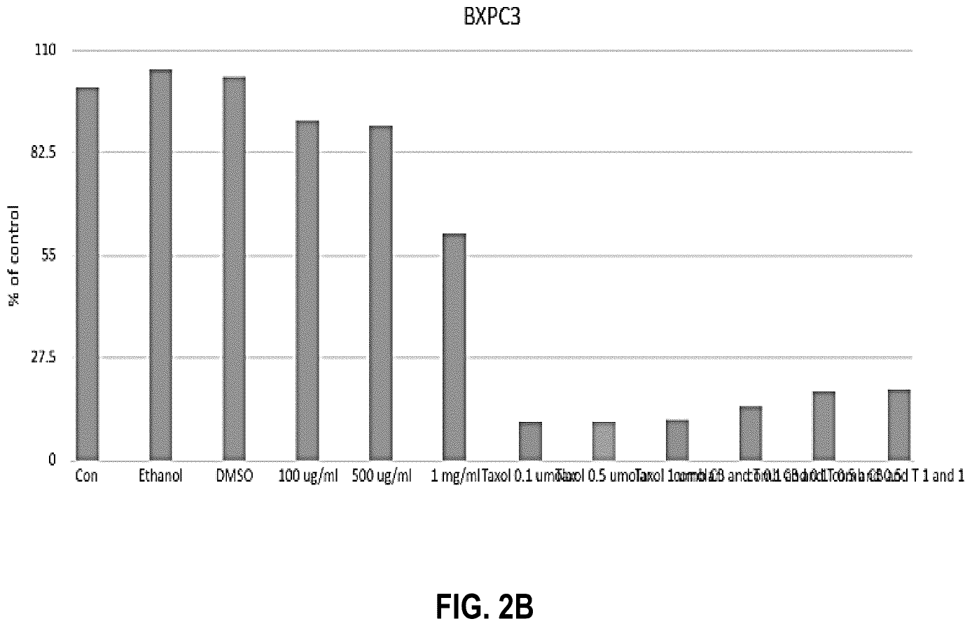

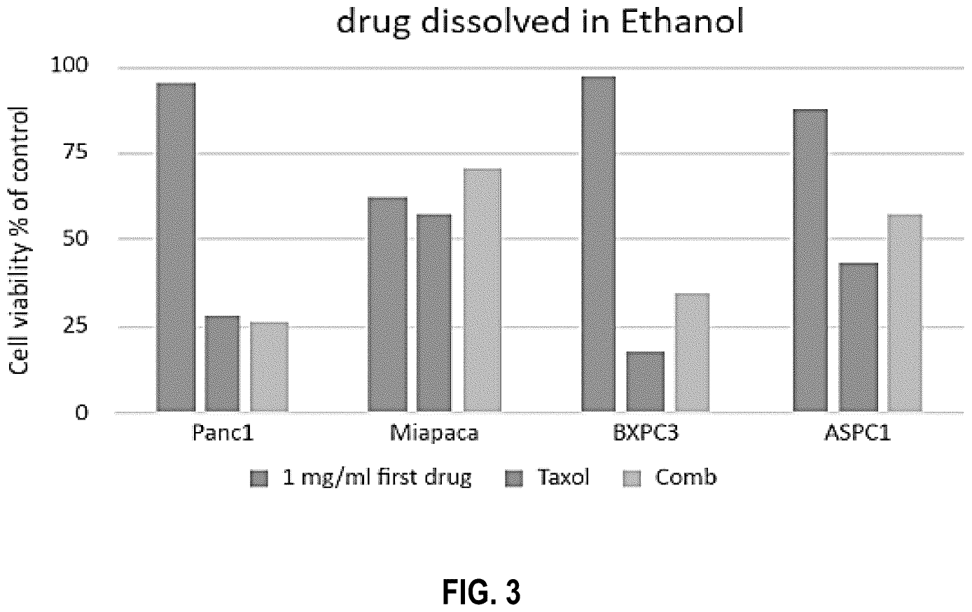

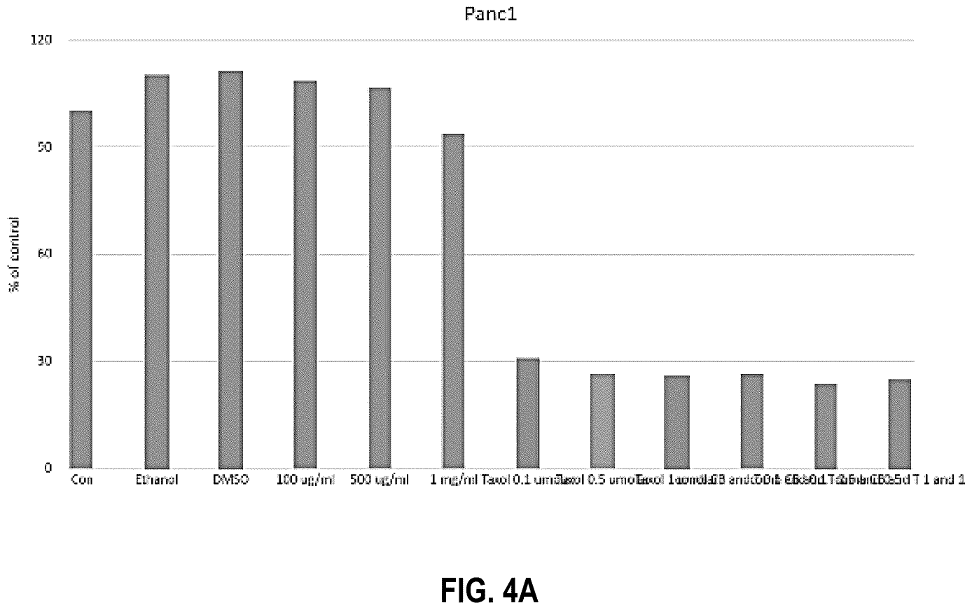

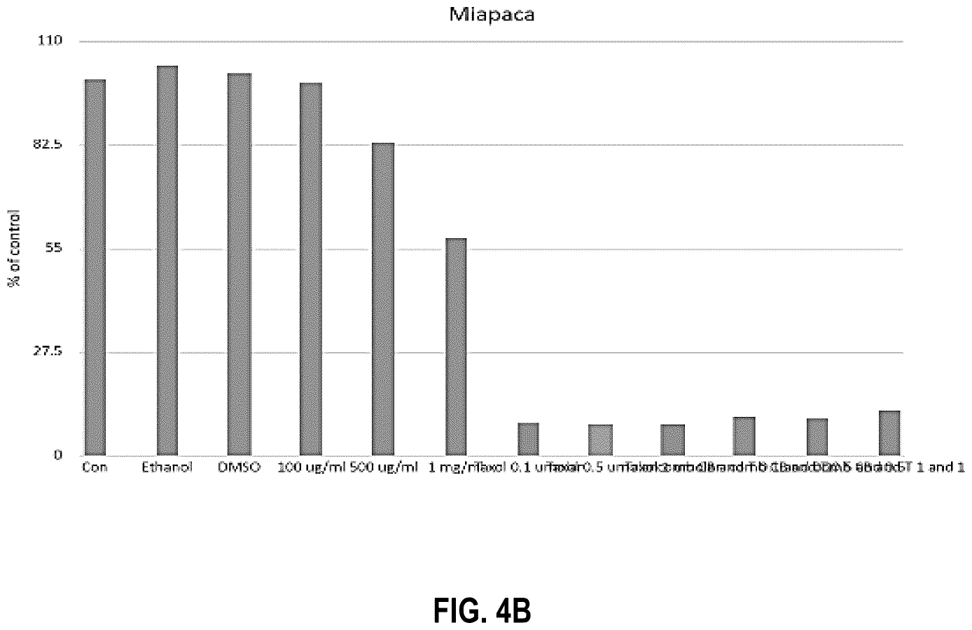

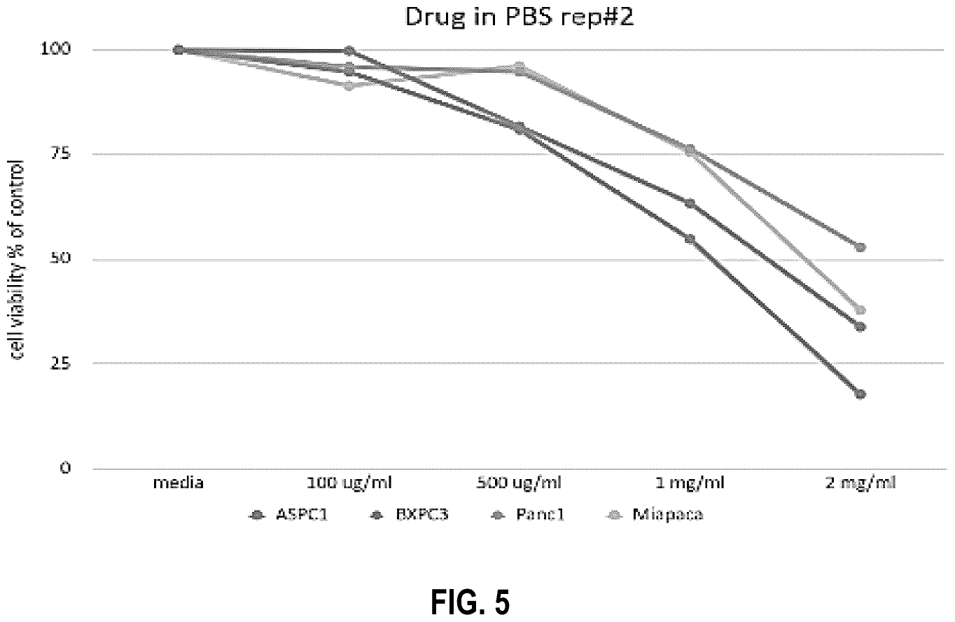

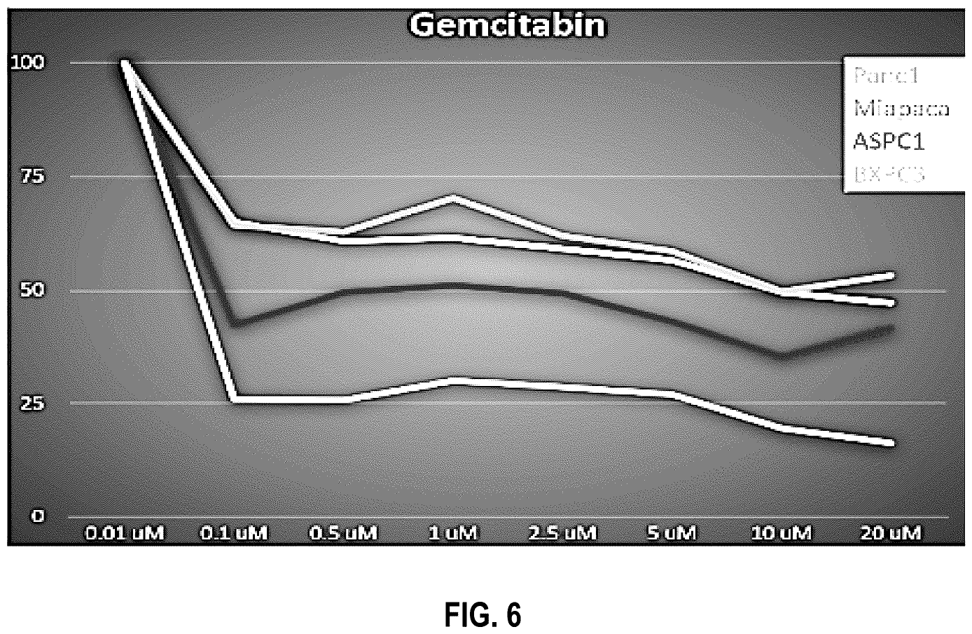

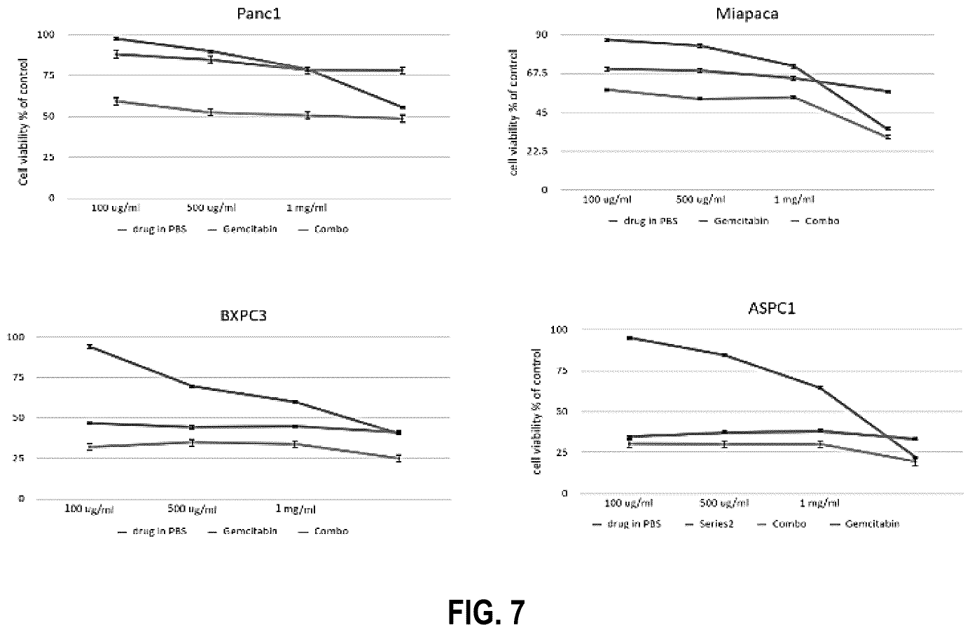

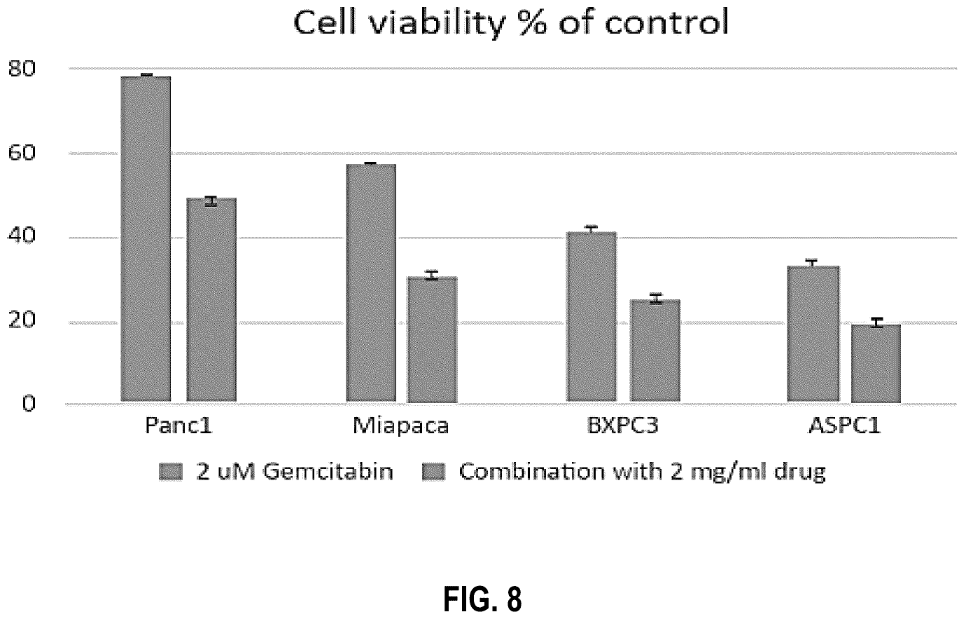

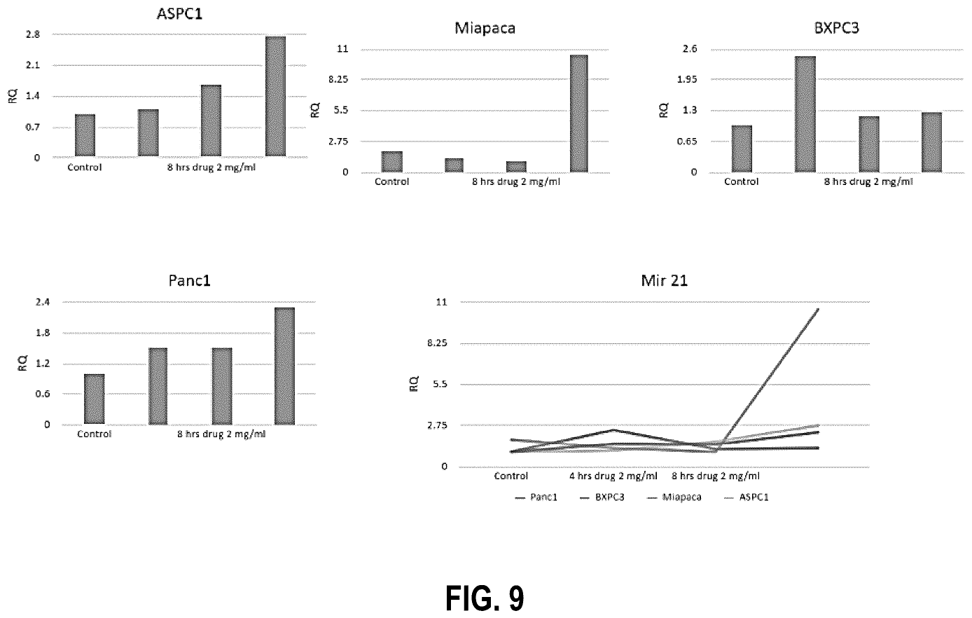

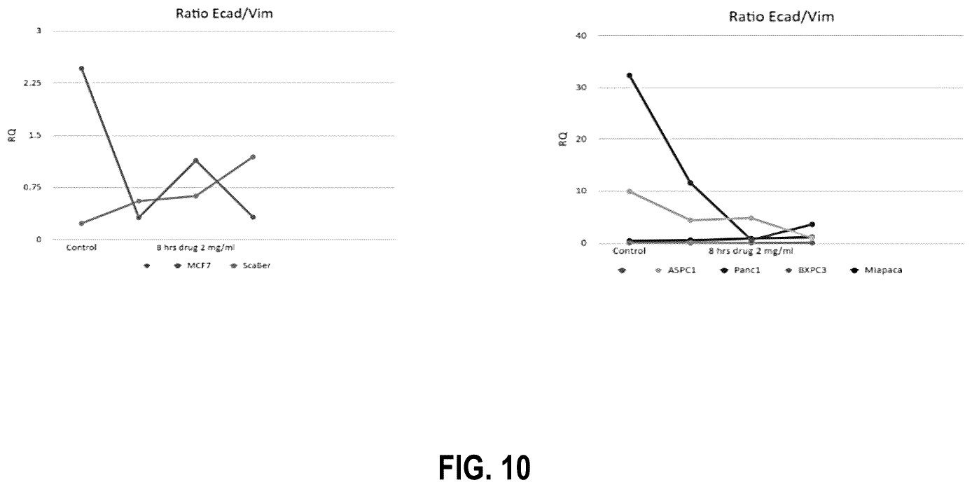

As outlined below in the examples some embodiments of the plant extracts, from Azadirachta indica (neem) specifically, can be used in the treatment of metastatic breast cancer (TABLES 1 and 2 and FIG. 1). This can be done by using the same or similar compositions that had been instead used as an anti-fertility treatment. In some embodiments, it would also be effective in cancer based on its applied use to attack hCG in the tumor cells.

In some embodiments, the methods and compositions according to the present disclosure utilizes a compound that has anti-fertility activity. In some embodiments, the compound has anti-human chorionic gonadotropin (hCG) activity and is an anti-hCG compound. In some embodiments, the anti-hCG compound has in vitro anti-hCG activity. In some embodiments, the anti-hCG compound has in vivo anti-hCG activity. In some embodiments, the anti-hCG compound has both in vitro and in vivo anti-hCG activities. In some embodiments, the anti-hCG activity blocks a sufficient amount of hCG so as to lessen the symptoms of the cancer. In some embodiments, the symptoms are lessened by at least 10, 20, 30, 40, 50, 60, 70, 80, 90, 95, 99, or 100%.

In some embodiments, the anti-hCG compound is a compound in Azadirachta indica (neem), Gossypium herbaceum, Trichosanthes kirilowii, Hibiscus rosa-sinensis (Malvaceae) Scutellaria barbata. In some embodiments, the anti-hCG compound is a synthetic analogue of a natural anti-hCG compound in Azadirachta indica (neem), Gossypium herbaceum, Trichosanthes kirilowii, Hibiscus rosa-sinensis (Malvaceae) Scutellaria barbata. In some embodiments, the anti-hCG compound has the ability to inhibit hCG in biological systems. In some embodiments, the anti-hCG compound is a compound that is isolated by one or more of the examples provided herein.

In some embodiments, the compound has one or more of an anti-hCG activity or an anti-fertility activity in vitro. In some embodiments, the compound has one or more of an anti-hCG activity or an anti-fertility activity in vivo. In some embodiments, the compound has one or more of an anti-hCG activity or an anti-fertility activity in vitro and in vivo.

In some embodiments, a method of preventing and/or treating a cancer is provided. In some embodiments, a method of preventing and/or treating a tumor is provided. In some embodiments, the method comprises identifying a subject with a tumor and/or a subject that is positive for a chorionic antigen.

In some embodiments, the subject is a mammal. In some embodiments, the subject is a human.

In some embodiments, the tumor or cancer is one or more of breast, liver, prostate, colon, bone, lung, kidney, uterus, pancreatic, or lymphocytes. In some embodiments, other tumors and/or cancers are also contemplated.

In some embodiments, the method comprises administering a compound to the subject in an amount that is adequate to prevent a cancer. Thus, in some embodiments, the method is preventative. In some embodiments, the method comprises administering a compound to the subject in an amount that is adequate to treat a cancer. Thus, in some embodiments, the method of treating is curative. In some embodiments, the amount that is administered is according to the amounts provided in the examples provided herein.

In some embodiments, the cancer comprises a tumor. In some embodiments, the cancer expresses a biomarker for a chorionic antigen. The expression of a biomarker for a chorionic antigen shows the presence of hCG in a cancer and hCG protects the cancer from being recognized or attacked by the immune system. In some embodiments, the method comprises administering an anti-hCG compound that inhibits hCG and/or blocks hCG thereby preventing hCG from protecting the cancer and allowing the immune system to recognize and attack the cancer.

In some embodiments, a method and/or composition to boost the immune system is provided. The method and/or composition to boost the immune system comprises an herbal substance as described herein. The method and/or composition may additionally comprise one or more treatment supplements such as multivitamins, immune supplements, and green shakes.

In some embodiments, the anti-hCG compound is isolated from a plant or produced synthetically. In some embodiments, the anti-hCG compound is a component of a plant. In some embodiments, the plant is one or more of Azadirachta indica (neem), Gossypium herbaceum, Trichosanthes kirilowii, Hibiscus rosa-sinensis (Malvaceae) or Scutellaria.

In some embodiments, the method uses an anti-hCG compound of the plant Azadirachta indica (neem). In some embodiments, the anti-hCG compound is a component in Azadirachta indica (neem). In some embodiments, the anti-hCG compound in Azadirachta indica (neem) is extracted from Azadirachta indica (neem) leaves. In some embodiments, the anti-hCG compound in Azadirachta indica (neem) is extracted from Azadirachta indica (neem) seeds. In some embodiments, the anti-hCG compound in Azadirachta indica (neem) is extracted from Azadirachta indica (neem) leaves according to the extraction method described in EXAMPLE 1. In some embodiments, the anti-hCG compound in Azadirachta indica (neem) is extracted from Azadirachta indica (neem) seeds according to the extraction method described in EXAMPLE 1. In some embodiments, the anti-hCG compound in Azadirachta indica (neem) is extracted from Azadirachta indica (neem) leaves and seeds. In some embodiments, when Azadirachta indica (neem) seeds are used, the solvent extraction as described in EXAMPLE 1 is used. In some embodiments, when Azadirachta indica (neem) leaves are used, the Soxhlet Extraction method as described in EXAMPLE 1 is used. In some embodiments, when both Azadirachta indica (neem) seeds and leaves are used, the solvent extraction method and the Soxhlet Extraction method as described in EXAMPLE 1 can be combined.

In some embodiments, extracts of Azadirachta indica (neem) can be administered for treatment of cancer. In some embodiments, the anti-hCG compound is in the Azadirachta indica (neem) extracts. In some embodiments, administering to a subject with metastatic breast cancer with multiple bone tumors an Azadirachta indica (neem) extract will result in a decrease in the cancer as determined by assaying for the levels of one or more cancer antigens (EXAMPLE 5; FIG. 1). In some embodiments, administering a subject with metastatic breast cancer with multiple bone tumors with Azadirachta indica (neem) extracts will allow for the discontinuation of one or more other anti-cancer treatments (EXAMPLE 5). In some embodiments, these can be administered in conjunction with existing chemotherapies to diminish their dosage and subsequent toxicity.

In some embodiments, the anti-hCG compound in Azadirachta indica (neem) is IRDNA. In some embodiments, the anti-hCG compound in Azadirachta indica (neem) is IRDNB. In some embodiments, IRDNA is extracted from an extract of Azadirachta indica (neem) leaves using the HPLC Fractionation method described in EXAMPLE 1. In some embodiments, IRDNB is extracted from an extract of Azadirachta indica (neem) leaves using the HPLC Fractionation method described in EXAMPLE 1. In some embodiments, IRDNA is administered to a subject. In some embodiments, IRDNB is administered to a subject. In some embodiments, both IRDNA and IRDNB are administered to a subject.

In some embodiments, the anti-hCG compound is a component in the plant Trichosanthes kirilowii (Cucurbitaceae, Dilleniidae). In some embodiments, the anti-hCG compound is a component in the root tuber of Trichosanthes kirilowii. In some embodiments, the root tuber of Trichosanthes kirilowii is used for extraction of the anti-hCG compound. In some embodiments, the anti-hCG compound extracted from Trichosanthes kirilowii is trichosanthin. In some embodiments, trichosanthin is a pure active crystalline protein. In some embodiments, trichosanthin is extracted from the root tuber of Trichosanthes kirilowii according to the method described in EXAMPLE 2. In some embodiments, an extract of root tuber of Trichosanthes kirilowii is administered to a subject. In some embodiments, the anti-hCG compound from the root tuber of Trichosanthes kirilowii is administered to a subject. In some embodiments, the anti-hCG compound in root tuber of Trichosanthes kirilowii is trichosanthin. In some embodiments, trichosanthin is administered to a subject.

In some embodiments, the anti-hCG compound is a component in the plant Hibiscus rosa-sinensis (Malvaceae) having an abortifacient effect, a contraceptive effect or a combination thereof (EXAMPLE 3). In some embodiments, the anti-hCG compound from Hibiscus rosa-sinensis (Malvaceae) is administered to a subject. In some embodiments, extracts of plants having one or more of anti-fertility, abortifacient or anti-hCG activities, can be used to inhibit or eradicate carcinogenic cells and tumors.

In some embodiments, the anti-hCG compound is a component in the plant Gossypium herbaceum. In some embodiments, the anti-hCG compound in Gossypium herbaceum is administered to a subject.

In some embodiments, the anti-hCG compound is a component in the plant Scutellaria barbata. In some embodiments, a proliferative effect of hCG on leiomyoma cells and myometrial cells is reduced (see, for example, EXAMPLE 4). In some embodiments, the proliferative effect of hCG on leiomyoma and myometrial cells is reduced by a component in Scutellaria barbata. In some embodiments, in hCG-treated leiomyomal cells, the expression of proliferating cell nuclear antigen, cyclin E and cdc2 are significantly reduced by a component in Scutellaria barbata. In some embodiments, the anti-hCG compound in Scutellaria barbata is administered to a subject.

In some embodiments, a composition for treatment of cancer is provided. In some embodiments, the treatment is preventative. In some embodiments, the treatment is curative. In some embodiments, the composition is administered to a subject in need thereof for prevention of a cancer. In some embodiments, the composition is administered to a subject in need thereof for prevention of a cancer.

In some embodiments, the subject has a propensity for developing a cancer. In some embodiments, the propensity for developing a cancer is determined based on genetic/familial history and/or based on a commercially available assay. In some embodiments, the subject has been diagnosed with a cancer. In some embodiments, cancer is one or more of breast, liver, prostate, colon, bone, lung, kidney, uterus, or lymphocytes. In some embodiments, other cancers, such as uterine and pancreatic cancer, are also contemplated.

In some embodiments, the cancer is an early stage, mid stage or late stage cancer. In some embodiments, the cancer is a combination of one or more of early, mid and late stage cancers. In some embodiments, the cancer is a Stage I, Stage II, Stage II or Stage IV cancer. In some embodiments, the cancer is a combination of one or more of Stage I, Stage II, Stage II or Stage IV cancer. In some embodiments, the cancer is in one location within the subject. In some embodiments, the cancer is in more than one location within the subject.

In some embodiments, the composition comprises an anti-hCG compound in one or more of Azadirachta indica (neem), Gossypium herbaceum, Trichosanthes kirilowii, Hibiscus rosa-sinensis (Malvaceae) or Scutellaria barbata. In some embodiments, the anti-hCG compound is administered in an amount that is adequate to prevent cancer in the subject. In some embodiments, the anti-hCG compound is administered in an amount that is adequate to treat cancer in the subject. In some embodiments, the cancer comprises tumor.

In some embodiments, the composition is a pharmaceutical preparation and/or composition comprising a pharmaceutically effective amount of an extract of one or more of Azadirachta indica (neem), Gossypium herbaceum, Trichosanthes kirilowii, Hibiscus rosa-sinensis (Malvaceae) or Scutellaria barbata and a physiologically acceptable carrier or delivery system. In some embodiments, the composition is a pharmaceutical preparation and/or composition comprising an active form of an anti-hCG compound in one or more of Azadirachta indica (neem), Gossypium herbaceum, Trichosanthes kirilowii, Hibiscus rosa-sinensis (Malvaceae) or Scutellaria barbata and a pharmaceutically acceptable carrier or delivery system. In some embodiments, the composition is a pharmaceutical preparation and/or composition comprising a pharmaceutically effective amount of the anti-hCG compound and a physiologically acceptable carrier or delivery system. In some embodiments, the composition is a pharmaceutical preparation and/or composition comprising an active form and pharmaceutically effective amount of the anti-hCG compound and a physiologically acceptable carrier or delivery system. In some embodiments, the composition comprises a pharmaceutical preparation/composition comprising a pharmaceutically effective amount of a natural form of the anti-hCG compound and a physiologically acceptable carrier or delivery system. In some embodiments, the composition comprises a pharmaceutical preparation/composition comprising a pharmaceutically effective amount of a synthetic analogue of a natural form of the anti-hCG compound and a physiologically acceptable carrier or delivery system. In some embodiments, the composition comprises a pharmaceutical preparation/composition comprising a pharmaceutically effective amount of a combination of a natural form and a synthetic analogue of the natural form of the anti-hCG compound and a physiologically acceptable carrier or delivery system. The physiologically acceptable carrier or delivery system may be inert or augment the effect of the anti-hCG compound.

In some embodiments, one or more of the following routes of administration are contemplated: parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal. In some embodiments, the composition to be administered can be formulated for delivery via one or more of the above noted routes.

In some embodiments, depending on the route of administration, the composition is formulated in a dosage form that is appropriate for the route of administration. In some embodiments, the route of administration is one or more of oral, parenteral (intramuscular, intraperitoneal, intravenous or subcutaneous injection), nasal, vaginal, rectal or sublingual. Other forms of administration such as via skin patches may also be used. Thus, the composition is formulated accordingly.

In some embodiments, when the route of administration is oral, the composition can be formulated as a solid dosage or a liquid dosage form.

In some embodiments, the solid dosage form is one or more of a capsule, a tablet, a pill, a powder or a granule. In some embodiments, the solid dosage form comprises an active form of the anti-hCG compound admixed with at least one inert pharmaceutically acceptable carrier. In some embodiments, the at least one inert pharmaceutically acceptable carrier is one or more of sucrose, lactose or starch. In some embodiments, the solid dosage form is a capsule, a tablet or a pill, the solid dosage form additionally comprises a buffering agent. In some embodiments, the solid dosage form is a tablet or a pill, solid dosage form additionally comprises an enteric coating.

In some embodiments, the composition is a liquid dosage form for oral administration, wherein the liquid dosage form is a pharmaceutically acceptable emulsion, a solution, a suspension, a syrup or an elixir. In some embodiments, the liquid dosage form comprises an inert diluent. In some embodiments, the liquid dosage form additionally comprises an adjuvant, wherein the adjuvant is one or more of a wetting agent, an emulsifying agent or a suspending agent. In some embodiments, the liquid dosage form additionally comprises one or more of a sweetening agent, a flavoring agent or a perfuming agent.

In some embodiments, the composition is a parenteral dosage form. In some embodiments, the parenteral dosage form is a sterile aqueous solution, a sterile non-aqueous solution, a suspension or an emulsion. In some embodiments, the sterile non-aqueous solution comprises a non-aqueous solvent or vehicle. In some embodiments, the non-aqueous solvent or vehicle is propylene glycol, polyethylene glycol, a vegetable oil, gelatin or an injectable organic ester. In some embodiments, the vegetable oil is olive oil or corn oil, and the injectable organic ester is ethyl oleate. In some embodiments, the parenteral dosage form additionally comprises an adjuvant, wherein the adjuvant comprises one or more of a preserving agent, a wetting agent, an emulsifying agent or a dispersing agent. In some embodiments, the parenteral dosage form is sterilized by one or more of filtration through a bacteria retaining filter, incorporating one or more sterilizing agents, irradiation or heating. In some embodiments, the parenteral dosage form is prepared immediately before administration using sterile water, a sterile injectable medium or a combination thereof.

In some embodiments, a composition for rectal or vaginal administration is a suppository. In some embodiments, the suppository may contain, in addition to the anti-hCG compound, an excipient. In some embodiments, the excipient can be cocoa butter, a suppository wax or both.

In some embodiments, a composition for nasal administration, sublingual administration or both are provided. In some embodiments, the compositions for nasal and sublingual administration are prepared with standard excipients well known in the art.

In some embodiments, a dosage amount of the anti-hCG compound is determined based on several parameters including, but not limited to, plant source, desired therapeutic effect, type of tumor and/or cancer, formulation used, route of administration and desired duration of treatment.

In some embodiments, the dosage amount of the anti-hCG compound is about 0.001 mg/kg to about 10 mg/kg of body weight per day. In some embodiments, the dosage amount of the anti-hCG compound is about 0.001, 0.01, 0.1, 1, 10, 100 mg/kg of body weight per day including any value and range above any of the preceding values and any value and range between any two of the preceding values. For example, in some embodiments, the dosage can be about 24 mg/kg of body weight per day (1500 mg QD (once per day) of EXAMPLE 1) of Neem extracts. In some embodiments, the dosage can be about 20, 21 22, 23, 24, 25, 26, 27, 28, 29 or 30 mg/kg of body weight per day.

In some embodiments, a subject of any age can be treated. In some embodiments, the age of a human subject can be about 10 years to about 70 years. In some embodiments, the age of a human subject is below 10 years. In some embodiments, the age of a human subject is above 70 years. In some embodiments, the age of a human subject is about 40 years.

In some embodiments, a subject of any weight can be treated. In some embodiments, the weight of a human subject can be about 100 lbs. In some embodiments, the weight of a human subject can be about 300 lbs. In some embodiments, the weight of a human subject can be about 140 lbs. In some embodiments, the weight of a human subject can be below 100 lbs. In some embodiments, the weight of a human subject can be above 250 lbs.

In some embodiments, a human subject may have one or more of other clinical, pathological, neurological, psychological, genetic conditions. In some embodiments, a human subject may be on one or more of other therapies for one or more of other clinical, pathological, neurological, psychological, genetic conditions.

In some embodiments, a human subject is a male. In some embodiments, a human subject is a female. In some embodiments, the duration of administration can be about 1 year to about 5 years. In some embodiments, the duration of administration can be shorter than about 1 year. In some embodiments, the duration of administration can be longer than about 5 years.

In some embodiments, the effectiveness of the anti-hCG compound is determined by tracking the tumor and/or cancer using one or more chorionic antigens or other antigenic and/or cancer-specific markers that are routinely used depending on the type of tumor and/or cancer. In some embodiments, the prevention and/or treatment of cancer is at least 75% effective. In some embodiments, the prevention and/or treatment of cancer is about 99% effective. In some embodiments, the prevention and/or treatment of cancer is at least 75% to about 99% effective. In some embodiments, the prevention and/or treatment of cancer is about 75, 80, 85, 90, 91, 92, 93, 94, 95, 96, 97, 98 or 99% effective at removing the detected cancer. In some embodiments, the treatment results in partial remission or complete remission of the cancer.

Any of the methods and/or compositions provided herein in regard to humans can also be provided to other mammals, for example, cow, sheep, dog, cat, pig, goat, guinea pig.

Although this invention has been disclosed in the context of certain embodiments and examples, those skilled in the art will understand that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes or embodiments of the disclosed invention. Thus, it is intended that the scope of the present invention herein disclosed should not be limited by the particular disclosed embodiments described above.

The present disclosure is further illustrated by reference to the following examples. These examples are provided for illustrative purposes, and are in no way intended to limit the scope of the invention.

### EXAMPLES

Azadirachta indica is a plant which is used in India and Africa for the treatment of the symptoms of malarial fevers and a variety of other illnesses, including skin diseases and inflammations. It has been studied as an anti-fertility agent both intrauterine and effecting sperm motility.

Azadirachta indica (neem) was studied as an anti-fertility agent, both individually and in conjunction with co-administration of the heterospecies dimer (HSD) hCG vaccine. Ref: Talwar G P, et al; Safety of intrauterine administration of purified neem seed oil (Praneem Vilci) in women & effect of its co-administration with the heterospecies dimer birth control vaccine on antibody response to human chorionic gonadotropin. Indian J Med Res. 1995 August; 102:66-70.

A crude, unpurified aqueous or alcoholic extract of the Neem leaves, bark and/or roots can be utilized for this symptomatic treatment. Neem seeds and oils are also used topically to treat skin infections and as insecticides and insect repellents.

The extraction of some antitumor substances from Neem bark has been reported by Pettit et al., Journal of Natural Products, Vol. 46 pp. 379-390 (1983) and Fujiwara et al., Carbohydrate Research 141, 168 (1985). These extracts were described as being useful for their toxic effects on cancer cells. However, these substances, like many other compositions used to treat cancer, are also toxic to noncancerous cells.

Similarly, several United States patents have reported Neem bark extracts which purportedly demonstrate antimitotic activity in fertilized sea urchin eggs and growth-inhibitory action against mouse sarcoma 180 ascites and solid tumors, and mouse L-5178Y cells. These patents are U.S. Pat. Nos. 4,515,785, 4,536,496 and 4,537,774 to Shimira et al.

The Neem tree (Azadirachta Indica) is a medicinal plant that grows wild in many parts of the world where the climate is warm, particularly in Asia and Africa. In Nigeria it is popularly called dogonyaro. All over Africa and Asia different parts of the Neem tree, leaves, bark and seed are employed as remedies for various human ailments. Neem medicinal products are widely used for treatment of skin diseases, inflammations, rheumatic diseases and fever. They are also employed extensively as antiparasitic agents in the treatment of helminthic and protozoal infections. In India, Neem oil and its isolates nimbidiol, nimbidin and diethylsulfides are used as anti-mycobacterial and antibacterial agents. In support of the diverse medicinal applications of Neem, extracts and isolated compounds from Neem exhibit various pharmacological activities. For instance, as an anti-inflammatory agent, an aqueous extract of Neem stem bark decreases both classical and alternative complement pathway activity, reducing complement-dependent responses, anaphylaxis, chemotaxis, opsonization and stimulation of macrophages and polymorphonucleoleucocytes. It also inhibits phorbol myristate acetate-stimulated chemiluminescence. Neem leaf extracts and isolated tetranortriterpenoids, gedunin and nimbolide, have been shown by various studies to inhibit the development of the human malarial parasite Plasmodium falciparum in vitro. Neem compounds, 7-acetyl neotrichilenone and 1,2-diapoxyazadiradione, were reported to inhibit the murine P-388 lymphocytic leukemia cell line with ED50 of 10 mg and 8.5 mg, respectively. Studies with various isolated Neem compounds have indicated that most of the medicinal and biological activity of the Neem is associated with the structural classes of limonoids, flavonoids, and macrolides, with sulfurated compounds playing minor roles.

Despite the extensive and centuries of use of Neem medicines, reports of toxicity in humans remain scanty. Experiments in mice suggest that various Neem compounds particularly oils, in large doses may produce toxicity. On the other hand, experiments in rats and dogs with nimbidin (a Neem compound) failed to demonstrate any systemic toxicity.

Human subjects given Neem oil, 7 grams per person, orally, or 1 gram by intramuscular injection experienced no local or systemic side effects. Thus, there is the expectation of low toxicity in its use as a drug and in handling the material. Interestingly, compounds isolated from the Neem leaves are azadiractin and related compounds which are very potent insect feeding repellents. This is a mechanism for the successful survival of this tree.

### Example 1A: Neem (Azadirachta Indica)

The extract from neem is administered to subject having cancer. The amount of isolated neem extract is increased by repeated doses until the cancer slows or stops spreading and/or is reduced in size.

### Description of Collection and Preparation of Neem Leaves

Fresh green leaves were collected from mature trees. The leaves were oven dried at 40°−50° C. over several days to a crisp. The dried leaves were then ground to coarse powder using a waring blender and stored in plastic bags at room temperature until used.

### Solvent Extraction of the Neem Seeds

For solvent extraction, a cold extraction procedure of crushed neem seeds in a sequence of hexane (NS 102/H), absolute alcohol (NS 102/E), distilled water (NS 102/W) was carried out. The yield of different fractions was found to be 76.7% for the hexane extract, 12.1% for the alcohol extract and 11.2% for the water extract of the total solvent extractable matter, which in total constituted 21.4% of the dry weight of seeds.

### Description of Soxhlet Extraction of Leaves

For extraction, the ground leaves was weighed and loaded into 33 mm Whatman cellulose thimbles, 15-20 μm per thimble. After loading, the thimble was inserted into the soxhlet apparatus and solvent mixture 25-50 ml made up to 50/50 acetone and distilled water was poured into the thimble to soak the ground leaves overnight. The following day, the soaked ground leaves was subjected to standard soxhlet extraction using additional 100 ml of the solvent mixture stated above. Extraction temperature was set at 70°−85° C. The extraction lasted for 24 hours or until the color of the eluate (solution flowing into the reservoir flask from the thimble) became colorless from its initial or starting green color. The extraction process yielded a deep green cloudy liquid-extract which was stored in sealed glass jars overnight in a refrigerator at 0°−8° C.

Recovery of Extracted Compounds from Liquid Extract

Buchi-type rotor-evaporation system was used to recover both the solvent and extracted compounds from the liquid extract under vacuum at 80° C.-90 C.

### Evaporation-Aided Separation Of Compounds IRC and IRAB

During the process of rotor-evaporation, fractions of the extracted compound continuously separated and precipitated on the sides of the rotor flask. As evaporation continued and after all the solvent were expelled, a brown oily liquid-residue remained. The oily liquid was poured out into a drying pan and dried to a paste overnight in air at 30°−40° C. The dried residue was recovered and code named IRC. To recover the precipitates on the sides of the rotor flask, 20-30 ml of acetone was poured into the flask and used to dissolve the precipitate. The solution was poured into a glass beaker and the acetone evaporated at room temperature over 1-2 days. The residue left after the evaporation of acetone was recovered and code named IRAB.

### HPLC Fractionation of IRAB and Isolation of IRDNA and IRDNB

IRAB was weighed and dissolved in DMSO at 37° C. Stock solution was prepared to contain 100 mg/ml. For analysis, the stock solution was diluted to desired concentration (10-50 mg/ml) and then fractionated by analytical HPLC using the following parameters.

A prepacked 300 mm×7.8 mm I.D. 10 ìM ì Bondapak C18 preparative column (Waters Assoc.) was used to chromatograph all compounds isolated in this study. The stationary phase of Bondak C18 is an octadecylsilyl bonded-phase packing material. The solid support medium contained in the column is capable of operating within a pH range of 2-8. It is thermally stable over a wide temperature range (<300° C.). Because of its excellent hydrolytic stability, various hydrophilic-hydrophobic mixed solvent systems are compatible with the column.

The mobile phase was 0.02M glacial acetic acid mixed with acetonitrile. Two high pressure pumps were used to deliver the mobile phase. Acetonitrile-0.02M acetic acid at a 35:65 percent ratio was used in an isocratic mode. Flow-rate was 1.5 ml/minutes. Column pressures ranged between 72-80 bar. All separations were performed at ambient temperatures. Sample volumes were introduced into the column through a continuous flow loop injected. Fractionated aliquots containing the chromatographed peaks were collected during multiple injections of the crude extracts. Detection of each peak was accomplished using an ultraviolet detector, set at 254 nm. Peak areas were measured by an on-line computing integrator.

Under the stated fractionation conditions, IRDNA and IRDNB appeared as separate chromatographic peaks eluting at variable retention times (RT) of 25-28 minutes for IRDNA and 29-31 minutes for IRDNB. The amount of IRDNA and IRDNB eluted also varies in a typical fractionation, in which 4 mg of IRAB was fractionated, 0.6 mg and 0.9 mg of IRDNA and IRDNB, respectively were obtained, representing 15 and 22.5% respectively of the fractionated IRAB.

### Example 2: Trichosanthes Kirilowii and Trichosanthin

Trichosanthin has been mentioned as effecting a lowering of hCG levels. A retrospective study aimed to assess the efficacy of trichosanthin (TCS) in combination with or without uterine arteries embolisation (UAE), uterine curettage and sac aspiration for the treatment of caesarean scar pregnancies (CSPs). 200 patients were enrolled at 4-17 weeks' postmenstrual age with suspected CSP. CSP was diagnosed based on serum β-human chorionic gonadotropin (B-hCG) level and transvaginal ultrasound. The patients were divided into TCS group and non-TCS group, who were treated with TCS and methotrexate, respectively, in combination with UAE and uterine curettage. TCS treatment had a success rate of 96.1% (50 of 52), similar to that in non-TCS group (98.6%, 146/148). Serum β-hCG levels on days 3, 5 and 7 in TCS group were significantly decreased. The complications were fever and pain, which were alleviated with symptomatic treatment. At follow-up, all 52 patients except one case with hysterectomy from TCS treatment group had resumed normal menstruation. In conclusion, TCS combined with bilateral UAE and uterine curettage is a safe and effective treatment for CSP, especially in patients with dangerously high serum β-hCG levels. (Ref: J Obstet Gynaecol. 2015 Aug. 24:1-5; Trichosanthin benefits the treatment of caesarean scar pregnancies; Li BL1, Qian W, Chen Q F).

A retrospective study of the effectiveness of trichosanthin (TCS), an active component isolated from the Chinese herb root tuber of Trichosanthes kirilowii on 140 cases of ectopic pregnancy with higher levels of β-human chorionic gonadotropin (β-hCG) managed with a single dose of TCS treatment. Trichosanthin has been used for medical treatment of ectopic pregnancy in China since the 1980s. This study was performed in a major teaching hospital in China. The mean pretreatment level of B-hCG in the TCS treatment group was 3387.57 IU/L. The success rate of TCS treatment was 85% (119 of 140) which was similar to methotrexate (MTX) treatment. In 86 women with a high level of β-hCG (over 2000 IU/L), the success rate was 80.08% when treated with TCS. Of this group, 26 women who had a high level of B-hCG (over 5000 IU/L) showed a success rate of 73%. The level of β-hCG on days 4, 7, and 10 in TCS group was significantly decreased. This study has shown that TCS may be an option for the medical treatment of unruptured ectopic pregnancy or an option for the treatment of ectopic pregnancy. (DJ Xiang et al., Trichosanthin, a Chinese Medicine for the Medical Treatment of Ectopic Pregnancy With High Levels of -hCG. Wuxi Maternity and Child Health Hospital Affiliated Nanjing Medical University, China. Reproductive sciences (Thousand Oaks, Calif.). 01/2012; 19 (5): 534-8).

The root tuber of Trichosanthes kirilowii (Cucurbitaceae, Dilleniidae) has been used together with six other traditional Chinese herbs in an ancient prescription for induced abortion. By a laborious process of elimination, a crude protein was subsequently isolated from the root tuber. The pure active crystalline protein, trichosanthin, was obtained by fractional precipitation of the crude protein with acetone, then by dialysis and lyophilization, and sometimes followed by further purification on CM-Sephadex C-50 columns. The molecular weight of trichosanthin is about 24,000 with an isoelectric point of pH 9.4. It does not contain carbohydrate or phosphorus, showing that trichosanthin is a simple protein.

Trichosanthin (TCS) is a 27 kDa protein extracted from the root tuber of the Chinese medicinal herb Trichosanthes kirilowii Maximowicz (Tian Hua Fen). TCS is synthesized as a preproprotein consisting of 289 amino acids, with a 23-residue signal peptide at the N-terminus and a 19-residue propeptide at the C-terminus. TCS is a type I ribosomeinactivating protein (RIP). It inactivates eukaryotic ribosomes by cleaving the N-glycosidic bond at adenine-4324 of 28S rRNA (Zhang and Liu, 1992) (REF: Toxicon 45 (2005) 683-689, Recent advances in trichosanthin, a ribosome-inactivating protein with multiple pharmacological properties, Pang-Chui Shaw). The proposed primary amino acid sequence of trichosanthin is given in Gu, Z. W., Qian, R. Q., Jin, S. W., Qian, W. W., Xu, S. Z., Zhang, L. Q., Zhang, X. L., Yao, Y. Z., Liu, Y. F., Zhu, S. Q., Cao, B. S., Wang, S. F., Wang, Q. H., Zhang, W. J., Liu, Y. Z., Fu, Y. H. and Wang, Y. (1984) Chemistry of trichosanthin. IV. The principle primary structure of trichosanthin. Acta Chimica Sinica 42, 943-945. It is a linear polypeptide of 224 amino acid residues with N-terminal Asp. Its C-terminal sequence determined by carboxypeptidase in combination with computer simulation is shown to be Asn-Asn-Met and Asn-Asn-Asn-Met-Ala in a ratio of 7:3. The sequence at residues 60-70 are yet to be completed, and those of residues 193-195 and 201-203 have not yet been determined unequivocally (Gu et al., 1984). X-Ray diffraction on the crystal structure of trichosanthin at 4 A resolution indicated that each trichosanthin molecule contains 8 segments of a-helix (approx. 85 amino acids accounting for 39% of the 224 amino acids) and 4 P-sheets comprised of 13 P-strands (approx. 70 amino acids, 32%) as well as some extended chains. Ref: (Pan et al., 1985a,b; Zhang et al., 1983a,b).

There are several reports on the success rates of clinical application of trichosanthin. A recent report on 100 cases of mid-term gestation by intra-amniotic injection of trichosanthin during 1982-1983 showed a success rate of 99%. The same study also reported that another 25 cases of retained dead fetus, 46 cases of missed abortion, 37 cases of hydatidiform mole and 37 cases of ectopic pregnancy were treated with trichosanthin intramuscularly in 1973-1982. The success rates were 96%, 93.5%, 86.5% and 86.5%, respectively. Abortion occurred usually on the 4th day after treatment and very little bleeding was encountered. The side effects of trichosanthin treatment such as fever and headache could be minimized by a simultaneous administration of dexamethasone (Jin, 1985).

Another group of clinical studies in 402 cases of therapeutic abortion with trichosanthin reported a success rate of 93.8% (93% complete abortion) (Liu et al., 1985). Among these 402 cases, 79.2% showed no undesirable effects, and 83.3% had no more bleeding than that of normal menstrual discharge. Laboratory examination showed no apparent change in hematological and urinary parameters after trichosanthin treatment, whereas follow-up studies showed that subsequent pregnancy was possible. It is widely accepted that trichosanthin acts by causing necrosis of the chorionic membrane, thus promoting the synthesis of prostaglandin in placental tissues. Structural damage of the trophoblastic tissues resulting in the impairment of the functions of the placenta was observed. Exploration on the pharmacological properties of trichosanthin excluded the possibility of termination of early pregnancy (Chang et al., 1979), but suggested an inhibitory effect on early pregnancy in mouse (Zhou et al., 1982). When trichosanthin was used in combination with other antifertility agents, including prostaglandin, total inhibition of early pregnancy was reported in man, mouse, rat and rabbit. (Jin et al., 1981; Liu et al., 1981a).

### Example 3: Hibiscus Rosa-Sinensis (Malvaceae)

Hibiscus rosa-sinensis has traditionally been used in Southeast Asia as an abortifacient, aphrodisiac, contraceptive, emmenagogue and uterine tonic. It has also been reported to be used to treat menstrual disorders and venereal diseases. Crude extracts of the flowers (Kholkute et al., 1976; Singh et al., 1982) and roots (Vasudeva and Sharma, 2008) show in-vivo post-coital anti-implantation activity in female rats (Kholkute and Udupa, 1976). This anti-implantation activity is probably due to the anti-estrogen activity of Hibiscus rosa-sinensis, as reported for extracts of the leaves (Nivsarkaretal., 2005) and flowers (A. Prakash, 1979; A. O. Prakash, 1979; Prakash etal., 1985, 1990). Furthermore, the abortive property of the flowers of Hibiscus rosa-sinensis could also be attributed to the growth inhibition of trophoblast cells (Zhao etal., 1998; Jiang, 2001), regression of the corpus luteum (Yanetal., 2000), or decreased secretion of human chorionic gonadotropin. (Hugo J. deBoer, Crystle Cotingting; Journal of Ethnopharmacology 151 (2014) 747-767; Medicinal plants for women's health care in southeast Asia: A meta-analysis of their traditional use, chemical constituents, and pharmacology).

The isolated compound HR-1 from Hibiscus rosa-sinensis L. is administered to subject having cancer. The amount of isolated HR-1 is increased by repeated doses until the cancer slows or stops spreading and/or is reduced.

### Example 4: Anti-Hcg Activity of Scutellaria Barbata

Scutellaria barbata D. Don (Lamiaceae) (SB) is a perennial herb which is natively distributed throughout Korea and southern China. This herb is known in traditional Chinese Medicine as Ban-Zhi-Lian and traditional Korean medicine as Banjiryun, respectively. SB has been used as an anti-inflammatory and antitumor agent. Some have determined the expression of cell cycle-related signal molecules for growth inhibition after hCG treatment by the herb SB in two different human myometrial smooth muscle cells (SMCs) and leiomyomal SMCs. Water-soluble ingredients of SB, myometrial SMCs and the leiomyomal cell lines were used in vitro. Uterine myomas often enlarge rapidly during pregnancy, implying that hCG may influences cell proliferation in uterine leiomyomata. We investigated the effects of SB on the cell proliferation and the expression of cell cycle-related proteins in these cells. Although hCG/LH receptor was present in both cultured myometrial and leiomyomal cells, as assayed by reverse transcription polymerase chain reaction analysis, treatment with hCG significantly increased cell proliferation in both myometrial and leiomyomal cells. However, SB reduced the proliferative effect of hCG in leiomyoma and myometrial cells, respectively. In hCG-treated leiomyomal cells, the expression of proliferating cell nuclear antigen, cyclin E and cdc2 was significantly reduced by SB treatment. These results suggest that SB reduced the hCG-promoted proliferation of myometrial and leiomyomal cells. (Tae-Kyun Lee; Dong-Il Kim; Young-Lim Song; Young-Choon Lee; Hyung-Min Kim; Cheorl-Ho Kim. Differential Inhibition of Scutellaria barbata D. Don (Lamiaceae) on hCG-Promoted Proliferation of Cultured Uterine Leiomyomal and Myometrial Smooth Muscle Cells; Immunopharmacology and Immunotoxicology, Volume 26, Issue 3 Jan. 2005, pages 329-342).

The extract from Scutellaria barbata is administered to subject having cancer. The amount of extract from scutellaria barbata is increased by repeated doses until the cancer slows or stops spreading and/or is reduced.

The extract can be prepared as outlined in IMMUNOPHARMACOLOGY AND IMMUNOTOXICOLOGY, Vol. 26, No. 3, pp. 329-342, 2004, Differential Inhibition of Scutellaria barbata, D. Don (Lamiaceae) on HCG-Promoted Proliferation of Cultured Uterine Leiomyomal and Myometrial Smooth Muscle Cells Tae-Kyun Lee, 1 Dong-Il Kim, 1 Young-Lim Song, 1 Young-Choon Lee, 2.

### Example 5: Anti-Hcg Activity of Momordica Charantia

Alpha-Momorcharin is another abortifacient protein (mol. wt 31,000) that can be purified from seeds of Momordica charantia (YEUNG et al., 1987). It is a basic glycoprotein with aspartic acid or asparagine at its N-terminal. In vitro studies have shown that it possesses biological activities similar to those of trichosanthin (LAW et al., 1983; TAM et al., 1985).

The plant extracts were subjected to acetone precipitation and further purified on CM Sepharose CL6B and Sephadex G50. Homogeneity of the proteins was confirmed by SDS-polyacrylamide gelelectrophoresis and immuncelectrophoresis.

Constituents1-3. Vicine, mycose, steroidal glucoside, momorcharaside A, B, cucurbitane triterpenoids, momordicines I and II, cycloeucalenol, spinasterol, stigmasterol, taraxerol, lophenol, momordicosides, diosgenin, thiocyanogen, 24-methylenecycloartenol, phenyl propanoids, carotenoids, squalene (seed essential oil), stigmastadien-3-beta-ol and glucoside.

Biological Activity4-6. Antimutagenic, abortifacient, antibacterial, antitumour, hyperglycemic, antiprotozoan, anthelmintic, antihyperglycemic, hypoglycemic, insecticidal, antilipolytic, CNS-depressent, cytotoxic, antispermatogenic, antifertility and spermicidal. Immature fruits gave several non bitter and bitter momordicosides. Fruits, seeds and tissue culture gave a polypeptide containing amino acids. Fruits also gave ‘-hydroxytryptamine, charantin (a steroidal glucoside), diosgenin, cholesterol, lanosterol and betasitosterol. Bitter principles are cucurbitacin glycosides. Chronic administration of the fruit extract to dogs led to testicular lesions with mass atrophy of the spermatogenic elements. The extract reduced the testicular content of RNA, protein and sialic acid as also the acid-phosphatase activity.

(Medicinal Plants of India, Indian Council of Medical Research, New Delhi.) The fruits and seeds yielded a polypeptide, p-insulin, which was considered similar to bovine insulin. The seed and fruit contain an inhibitor of HIV, (Momordica anti-HIV-protein) which exhibited antiviral and antitumour activity in vitro. Another protein, MRK-29found in the seed and fruit of a smaller var. of Bitter Gourd found in Thailand, was found to inhibit HIV reverse transcriptase and to increase tumor necrosis factor (TNF). (Planta Med, Natural Medicines omprehensive Database. The seeds yield alpha- and betamomorcharins (glycoproteins). When these glycoproteins were co-cultured with isolated hepatocytes, morphological changes in hepatocytes were observed, indicating hepatotoxicity. Another principle with antilipolytic and lipogenic activities, found along with the alpha- and beta-momorcharin in the seed extract, did not show toxic effect.

### Example 6: Clinical Case

This is a clinical study on a 40 year old female suffering from metastatic breast cancer with multiple bone tumors
S.P. is a 40 year old female
5′ 11′
138 lbs
married
DOB: Nov. 25, 1970
Born: Richmond, IN
CC: She presents with Stage IV breast cancer
Dates: Treatment with Neem began in April 2011 and continued until April of 2012
She has complicating issues with current treatment of a diagnosis which involve pain, anxiety, and impairment of overall health.
History: Kidney disorder IGA nephropathy, hashimoto's thyroiditis
Medications: oxcycodon, acetaminophen, temazepam, gabapentin, lexapro, synthroid, xeloda, vitamin b's, raw heart, liver & lung, multivitamin, immune supplement, green shakes, silymarin, melatonin, 5HTP

### Past Medical History:

IBS as a teenager; fibroid tumors in breasts in 20s; anxiety/depression during 20s & 30s; childbirth at 30 & 33; diagnosed at age 30 with hashimotos disease given thryoid supplement; diagnosed at age 33 with IGA nephropathy; 36 double mastectomy, chemo, reconstruction; 37 radiation to spine been in treatment with targeted steroid hormones and currently oral chemo; 37 ovaries removed broken hand and hand surgery at age 39; septic shock at age 39; acute renal failure at age 36 & 39 under no kidney treatments to date; wisdom teeth 1997; shoulder 1995; double mastectomy 2006; reconstruction 2006 & 2007 (saline implants); ovaries removed 2007.

### Family History:

Father—Type 2 diabetes; abdominal cancer-cured by a homeopathic Dr, pancreatic cancer; in 70s developed heart issues needed heart surgery but was too weak; passed away January 2010 of malnutrition associated with cancer treatment.

Mother—heart; thyroid; cataract.

Left breast origin of cancer. Double mastectomy. Estrogen and progesterone positive.
In October ovaries removed.
Spread to bones with 109 tumors detected. And 15% liver affected.
History of Irritable Bowel Syndrome as a teenager. Sent to a rheumatologist.
Before ovaries removed had pms, anger . . . emotional (premenstrual syndrome with symptoms of anger and heightened emotions).
On first doing chemo was suicidal.
Patient felt isolated and lonely in bed because of pain.
Was very active in work previously.
P: Patient placed on Neem extracts 1500 mg QD
Chemotherapy was discontinued in June 2011

TABLE 1
LAB RESULTS DURING TREATMENT

| | Jun. 30, 2011 | Jul. 25, 2011 | Aug. 22, 2011 | Sep. 19, 2011 | Nov. 14, 2011 | Dec. 12, 2011 | Dec. 29, 2011 | Jan. 13, 2012 |
|---|---|---|---|---|---|---|---|---|
| CEA | 17.4 | 6.3 | 4.8 | 4.1 | 3.7 | 5.1 | 6.9 | 20.8 |
| CA2729 | 135 | 119 | 71 | 36 | 22 | 30 | 30 | 62 |
| | 13% | 5% | 7% | 11% | 17% | 17% | 23% | 34% |
| Cellsearch | | | | | | | 325 | |
| PET scan | | | | | | | | |
| Creatinine | 1.19 | 0.86 | 0.83 | 0.82 | 0.78 | 0.92 | 0.8 | 0.98 |
| BUN Ratio >22 | 13.4 | 15.1 | 18.1 | 15.9 | 15.4 | 12 | 12.5 | 13.3 |
| Alkaline | | | | | | | | |
| Phosphatase | 232 | 135 | 101 | 82 | 64 | 66 | 73 | 94 |
| >115 | | | | | | | | |
| AST (SGOT) >30 | 61 | 24 | 24 | 24 | 24 | 24 | 20 | 29 |
| ALT (SGPT) >40 | 52 | 16 | 15 | 16 | 13 | 14 | 10 | 22 |
| mth over mth | | | | | | | | |
| CEA | | −64% | −24% | −15% | −10% | 38% | 35% | 201% |
| CA2729 | | −12% | −40% | −49% | −29% | 36% | 0% | 107% |
| Creatine | | −28% | −3% | −1% | | 18% | −13% | 23% |
| Alkaline | | −42% | −25% | −19% | | 3% | 11% | 29% |
| Phosphatase | | | | | | | | |
| AST (SGOT) | | −61% | 0% | 0% | | 0% | −17% | 45% |
| ALT (SGPT) | | −69% | −6% | 7% | | 8% | −29% | 120% |

TABLE 2
CANCER ANTIGENS (CEA AND CA2729)

| | Feb. 10, 2012 | Mar. 9, 2012 | Jun. 1, 2012 | Jul. 2, 2012 | Aug. 2, 2012 | Aug. 27, 2012 | Sep. 24, 2012 |
|---|---|---|---|---|---|---|---|
| CEA | 31.8 | 27.29 | 14 | 18.9 | 20.4 | 19.1 | 21.6 |
| CA2729 | 74 | 106 | 58 | 70 | 74 | 62 | 74 |
| | 43% | 26% | 24% | 27% | 28% | 31% | 29% |
| cellsearch | 3 | | | | 6 | | 75 |
| PET scan | | | | | | | |
| creatinine | 0.89 | 0.94 | 0.92 | 0.87 | 0.86 | 0.85 | 0.97 |
| BUN Ratio >22 | 27 | 16 | 21.7 | 21.8 | 19.8 | 20 | 14.4 |
| Alkaline Phosphatase | 124 | 98 | 58 | 76 | 80 | 63 | 62 |
| >115 | | | | | | | |
| AST (SGOT) >30 | 28 | 29 | 24 | 29 | 29 | 30 | 34 |
| ALT (SGPT) >40 | 19 | 24 | 16 | 19 | 19 | 17 | 20 |
| mth over mth | | | | | | | |
| CEA | | −14% | | 35% | 8% | −6% | 13% |
| CA2729 | | 43% | | 21% | 6% | −16% | 19% |
| creatine | | 6% | | −5% | −1% | −1% | 14% |
| Alkaline Phosphatase | | −21% | | 31% | 5% | −21% | −2% |
| AST (SGOT) | | 4% | | 21% | 0% | 3% | 13% |
| ALT (SGPT) | | 26% | | 19% | 0% | −11% | 18% |

In summary, the patient was documented to have a partial remission of both liver, lymphatic and bone tumors. The patient's Chorionic antigen 2729 was reduced from a peak of 135 to 22 in a period of 4.5 months. This measures the reduction in a breast tumor marker. In the same period her Alkaline phosphotase was reduced from a high of 232 (norm is <115) which is an indicator in bone tumours to a norm of 62. In the first month of treatment the patient's Alkaline phosphotase declined 42%. The patient's liver enzymes AST and ALT were reduced by 61% and 69% respectively. The patient's CEA (carcinoembryonic antigen) dropped by 64%.

### Example 7: In Vitro Study on Cancer Cell Lines

Effect of Trichosanthin and Scutellaria barbara on the Viability of Pancreatic Cancer Cell Lines

A study on cancer cell lines was conducted at Wake Forest University, Division of Internal Medicine/Section on Hematology & Oncology, Winston-Salem, North Carolina by Pierre Triozzi M. D. and Mitra Kooshki, MS Lab manager at the request of Dr. Andrew Lange. Samples were provided by Dr. Lange.

The study was blinded by Dr. Lange. Analysis was contributed by Muhammad M Rahman.

Effect of CB with Taxol on the Viability of Pancreatic Cancer Cell Lines

Four immortalised pancreatic cancer cell lines were tested for their drug response to Taxol and CB alone, or together. MTT assays were performed on 10,000 cells. The assay measures cell viability, cytotoxicity, and proliferation. The drugs were dissolved in ethanol and untreated cell controls are given as 100% cell viability. Ethanol alone killed between 10-25% of the cell lines, with Miapaca the least affected and Panc1 the most affected.

CB treatment alone had the greatest effect on Miapaca cells, killing nearly 40% of cells at 1 mg/ml. Miapaca cells were less affected by Taxol compared to the other cell lines, but there was 55% cell viability, which suggests that Taxol alone is more effective than CB alone. Combined treatment did not show a reduction in cell viability, instead there was an increase in cell viability.

ASPC1 treated with CB alone showed greater cell viability than compared with ethanol, which suggests that CB has no effect on these cell lines. Taxol treatment at 1 μM showed a 60% reduction of cell viability and when combined with CB, there was no significant difference given that CB alone appears to be ineffective in ASPC1 cell lines.

When treated with CB alone, BXPC3 cells showed no cytotoxicity and appear to have improved proliferation. Treatment with 1 μM Taxol alone resulted in a 80% reduction in cell viability and clear response to Taxol concentration was observed. Combined treatment of CB with Taxol was less effective against BXPC3 cells, with CB possibly countering the effects of Taxol.

Similar to BXPC3 cells, Panc1 cells treated with CB showed improved cell proliferation compared to ethanol treatment alone, with a cell viability of over 90%. However, treatment with Taxol had a strong effect on cell viability with a reduction of approximately 70%. Combined treatment of CB with Taxol had no significant change on cell viability compared with Taxol treatment alone.

Collectively, these data show consistent efficacy of Taxol treatment in at least 40% of all cell lines. The strongest effect of Taxol alone was seen in Panc1 cells. Only Miapaca cells responded to CB treatment whereas the other cell lines appear to be resistant. Furthermore, when combined with Taxol, there was no significant effect on Miapaca cells despite being the only cells that were responsive to CB treatment.

The four pancreatic cancer cell lines should be characterised in terms of their behaviour in culture. The proliferation rate, phenotype, colony formation profile, proportion of epithelial-to-mesenchymal (EMT) migratory cells, and other tumour-related characteristics should explored as they are often linked to therapy resistance, secondary tumour formation, and recurrence.

An important factor that is often ignored in drug treatment studies is how the drug affects the different cell populations. It is well documented that cancer stem cells are more resistant to chemotherapy than the bulk epithelial population, therefore, it would be useful to determine how these population respond. For instance, if a drug is effective in killing bulk population but not the cancer stem population, the surviving population becomes enriched leading to the accumulation or more cells that have a higher metastatic potential. Furthermore, longer drug exposure studies would be useful to establish if the surviving cells develop a resistance and cells should also be monitored after treatment is stopped for changes in behaviour. This would be particularly interesting in tumour colony formation assays or 3D tumour models.

### Introduction of Two Experimental Drugs:

Drug 1: Trichosanthin (an isolated compound from Trichosanthes kirilowii)
Drug 2: Scutellaria barbarata tincture

TABLE 3

| Drug only | Panc1 | Miapaca | BXPC3 | ASPC1 |
|---|---|---|---|---|
| 1 mg/ml | 95.7 | 61.96 | 96.98 | 87.52 |
| first drug | | | | |
| 1 mg/ml | 83.53028 | 77.25892 | 65.34929 | 61.44573 |
| second | | | | |

This graph compares the use of the drug, Taxol, and both combined, when dissolved in ethanol. The most effective option to kill the cell lines is with Taxol, combination with the drug did not reduce cell viability.

These graphs highlight the best combination of drug and Taxol for killing the cancer cell lines. For BXPC3, Taxol alone was the most effective treatment. For ASPC1, Panc1, and Miapaca. The combination of drug with Taxol was most effective.

The graph shows that the use of the drug at higher concentration is more effective in killing the cancer cell lines. The effectiveness of the drug at 2 mg/ml was considerably higher than 1 mg/ml.

Gemcitabin on the cell lines shows that beyond 0.1 μM, there is no improvement in the drug effectiveness. Panc1 and Miapaca appear to be the most resistant cell lines, followed by ASPC1, then BXPC3. A possible reason for this could be the aggressiveness of the cell lines.

These graphs show that overall, combination of the drug and Gemcitabin (20 μM) are most effective for killing the cell lines up to 1 mg/ml. The drug is most effective and with increasing concentration. The addition of Gemcitabin kills more of the cells, but increasing concentration does not have a major effect (as seen on the previous graph). However, at the fourth datapoint (2 mg/ml) the drug alone is able to reach the same level of effectiveness as using the combination.

TABLE 4

| | | Combination with |
|---|---|---|
| n = 3 | 2 uM Gemcitabin | 2 mg/ml drug |
| Panc1 | 78.35449 | 48.76401 |
| Miapaca | 57.32845 | 30.74018 |
| BXPC3 | 41.29571 | 25.21561 |
| ASPC1 | 33.22361 | 19.50957 |

Following on from the previous set of graphs, this highlights the most aggressive to least aggressive cell lines. Here, the combination of drugs shows a ~50% increase in effectiveness compared to Gemcitabin alone. Again, 20% of cells survive the combination drug treatment. By looking at this data and graph in particular, there is a synergistic effect.

Here, the drug reduces miR-21 expression for both cancer cell line. For MCF-7, there was the 30-fold increase compared to a normal breast cell line. Therefore the drug 1 is able to reduce miR-21 expression levels down to baseline.

These graphs show that compared to MCF-7 and ScaBer, the drug have an opposite effect on miR-21 expression in the cancer cell lines. What is interesting about this data is that the surviving cells are most likely the 25% of drug resistant cells and these have an increase in miR-21 expression.

The final two graphs, show that MCF-7, ASPC1, and Miapaca/Panc1 (both are the same colour on the graph), the drug causes the expressions of ECAD to decrease and VIM to increase, therefore moving towards an EMT phenotype. For BXPC3 and Miapaca/Panc1, there appears no change, but this could depend on the baseline levels of ECAD and VIM. For ScaBer, the cells seem to become more epithelial so it is likely the drug can prevent metastasis.

Breast cancer is characterised by an elevated capacity for tumour invasion and lymph node metastasis, but the cause remains to be determined. Recent studies suggest that microRNAs (miRNAs) can regulate the evolution of malignant behaviour by regulating multiple target genes. A key oncomir in carcinogenesis is miR-21, which is consistently upregulated in a wide range of cancers. miR-21 expression is increased in breast cancer and targets STAT3, which may act as a double-response controller in breast cancer. We have generated some preliminary data showing that Drug A (Trichosanthin) is able to kill a large percentage of immortalised breast cancer cells, also correlating with a reduction in mir-21 expression. Drug A in combination with Taxol, was successful in killing large proportions of pancreatic cancer cell lines.

Research Strategy: Optimize the Efficacy of Drug a with or without Taxol on Cancer Cell Lines

Cancer stem cells (CSCs) are a small and undifferentiated stem-like cell subpopulation within the tumor heterogeneous cell types. CSCs contribute to cancer initiation, metastasis, resistance, and cancer relapse. CSCs are similar to normal stem cells as they self-renew and give rise to various cell types. Regarding tumorigenesis, CSCs have the capacity for sphere formation. CSCs appear to signal to neighboring cells to provide nutrients, cooperate in their evasion from the immune system, and create a microenvironment that favors tumor growth. CSCs have enhanced survivability in a tumor environment of reduced oxygen and nutrients, and increased resistance to chemo, immune, and radiotherapies. These are features that tumors use to escape from treatment and promote recurrence; therefore, it is vital to consider this population of cells to improve cancer therapy.

### Related Literature

Additional examples of plants and their constituents which have shown anti-fertility and abortifacient effects which could have action against hCG in cancer cells are as follows:

### Animal Activity References

### A. Anti-Implantation Activity

1. Abrus pricatorius Linn. SD Abridine 1 mg/animal 2-5 Rat 100 Zia-ul-Haque et al. (1983a,b)
2. Achyranthes bidentata RT Saponins 218 1-10 Mice—Zhu (1982), Zhu and Che (1987)
3. Adiantum capillus PL Isoadiantone—Rat—Murthy et al. (1984)
4. Ananas comosus Merr. LF 5-stigmastane-3,5,6-triol 3-mon 40 6-7 Mice 100 Pakrashi and Chakrabarty (1979); Sitosterol 30 1 Mice 93 Pakrashi and Basak (1976); Ergosterol peroxide 30 6-7 Mice 100
5. Aristolochia indica Linn. RT Aristolochic acid 100 1, 6 or 7 Mice 100 Pakrashi and Chakrabarty (1978); p-Coumaric acid 50 6 Mice 100 Pakrashi and Pakrashi (1978)
6. Butea monosperma (Lamb) Kuntz
   B. Isolated Chemical Constituents from Plants with Antifertility Potential.
1. No.; Name of plant Part used; Active principle; Dose mg/kg; po Days
2. Ref: SD Butin 20 1-5 Rat 90 Porwal et al. (1988)
3. Centella asiatica LF Isothankuniside and BK Compound [methyl-5-hydroxy-3,6-diketo-23 (or 24)-norurs-12-en-28-oat]—Mice Consisent Dutta and Basu (1968)
4. Datura quercifolia—Daturalactone (DQ1) 100 Jan. 7, 1973.3 Chandhoke (1978), Chandhoke and Gupta (1978a)
5. Dictamnus albus RB Fraxinellone—1-10 Rat—Woo et al. (1987)
6. Embelia ribes Burm.f. BR Embelin 100 1-5 Rat 100 Kholkute et al. (1978); Bhargava and Dixit (1985)
7. Foeniculum vulgare SD Anethole 500 1-5 Rat 60 Seshadari and Pillai (1981)
8. Heliotropium indicum—n-hexacosanol, sitosterol, stigmasterol, chalinasterol, Campesterol 500—Rat 40 Andhiwal et al. (1985)
9. Marsdenia koi PL Marsdekoside A and B—Rat—Yuan et al. (1991)
10. Murraya paniculata RT Yuehchukene 3 1-2 Rat Potent Kong et al. (1985a)
11. Piper longum Linn. RT Piperine 150 1-7 Rat 60 Gupta et al. (1977), Kholkute et al. (1979)
12. Plumbago zeylanica Linn. PL Plumbagin 20 1-5 Rat 83 Chowdhury et al. (1982), Premakumari et al. (1977)
13. Randia dumetorum Lamk. SD Oleanolic acid-3-glucoside 100 1-5 Rat 100 Pillai et al. (1982)
14. Ruta graveolens Linn. RT, SB, LF Chalepensin 36 1-8 Rat 80 Kong et al. (1989)
15. Striga lutea PL Acacetin, Luteolin 5-25 1-4 Rat, Mice Significant Hiremath et al. (1990); Hiremath and Rao (1990)
16. Vicoa indica (L.) DC PL Vicolide B 50 8-14 Rat 100 Susan et al. (1985), Alam et al. (1992a,b) Vicolide D 200 8-14 Rat 71 Alam et al. (1992a,b)
17. Vitex negundo Linn. SD 5,7,3-trihydroxy-6,8,4-trimetho flavones 100 4-6 Mice 100 Bhargava (1984)

### C. Abortifacient Activity

1. Aristolochia indica Linn. RT Methyl aristolate 60 6, 7 Rat 100 Xu and Gao (1986)
2. Daphe Sp. F L Yuanhuatine 50 g-Monkey Significant Hu et al. (1984)
3. Daphne genkwa RT Yuanhuacine 70-80 g—Woman Significant Ren-Sheng and Yi-Sheng (1986)
4. Momordica charantia Linn. SD and Momorcharins—Mice Significant Yeung et al. (1986)
5. Momordica cochinchinensis RT Momorcochin—Mice-Yeung et al. (1988)
6. Piper sp.—Piperine—8-12—Significant Kholkute et al. (1979), Piyachaturawat et al. (1982)
7. Plumbago zeylanica Linn. PL Plumbagin 10 5-11 Rat 75 Bhargava and Dixit (1985), Premakumari et al. (1977) 50 6-9 Rat Significant

### D. Contraceptive Activity

1. Aehonychon purpurea-caemleum—Lithospermic acid Mats et al. (1982)
2. Androsace septentrionalis—Triterpene glycoside 100—Mice, rat—Mats et al. (1984), Mats and Savchenko (1986)
3. Citrus aurantium Peel Cirantine 0.75—Rabbit—Ghosh et al. (1955)
4. Ferula jaesochkeana PX Ferujol 0.6 1-5 Rat 100 Singh et al. (1985), Prakash et al. (1991) Ref: Dinesh Kumar, Ajay Kumar, Om Prakash, Journal of Ethnopharmacology 140 (2012) 1-32 Potential antifertility agents from plants: A comprehensive review.

### D. Constituents Specific to Neem

1. Triterpenoids and steroids isolated from neem (A. indica)
2. Name of constituents; Chemical formula; Melting point (° C.); Isolated from
   a. Nimbin C30H36O9 205 Oil, trunk and root bark
   b. Nimbinin C28H34O6 202 Oil, trunk and root bark
   c. Nimbidic acid (Salannic acid) C26H34O7 228 Oil
3. Salannin C34H44O9 167 Oil
4. Deacetyl Nimbin C28H34O8 208 Seeds and bark
5. Nimbolide C27H30O7 245 Leaves
6. Meliantriol C30H50O5 176 Oil
7. Azadirone C28H36O4 192 Oil
8. Epoxyazadiradione C28H34O6 202 Oil
9. Azadiradione C28H34O5 205 Oil
10. Gedunin C28H34O7 218 Oil
11. 7-Deacetyl Gedunin C26H34O6 259 Oil
12. Meldenin C28H38O5 240 Oil
13. Salannin-lactone C34H44O10 244 Oil
14. Nimbin-lactone C30H36O10 184 Oil
15. C35H44O16 155 Seeds
16. Vepinin C28H36O5—Oil
17. Nimbolin A C39H46O8 180 Trunk wood
18. Nimbolin B C39H46O10 243 Trunk wood
19. Nimbidinin C26H34O6 282 Oil
20 Vilasinin C26H36O5 255 Leaves
21. Nomolin C28H34O6 205 Fruits
22. Nimolicin C28H34O5 166 Fruits
23. 17-Hydroxy-azadiradione C28H34O6-Fruits
24. 17β-Hydroxy-azadiradione C28H34O6 177 Fruits
25. 17-Epi-azadiradione C28H34O5 205 Fruits
26. 1α-Methoxy-1,2-dihydroepoxy-azadiradione C29H38O7 235 Seeds
27. 1β, 2β-Diepoxy-azadiradione C28H34O7 110 Seeds
28 7-Acetylneotrichilenone C28H36O5 208 Seeds
29. 7-Deacetyl-7α-benzoylazadiradione C33H36O5 Amorphous Seeds
30. 7-Deacetyl-7α-benzoylepoxy-azadiradione C33H36O6 Amorphous Seeds
31. 7-Deacetyl-7α-benzoylgedunin C33H36O7 278 Seeds
32. Nimbinene C28H34O7 134 Oil, leaves and bark
33. 6-Deacetyl nimbinene C26H3206 141 Oil, leaves and bark
34. Nimbandiol C26H32O7 121 Oil, leaves and bark
35. 6-o-Acetyl-nimbandiol C28H34O8 178 Oil
36. 3-Deacetylsalannin C32H4208 214 Oil
37. Salannol C32H44O8 208 Oil
38. 1,3-Diacetyl-vilasinin C30H4007 157 Oil
39. Nimocinol (6α-Hydroxy-azadirone) C28H36O5 130 Leaves
40. β-Sitosterol C29H50O 140 Blossom, leaves and wood oil
41. β-Sitosterol-β-D-glucoside C35H6006 283 Blossom, leaves and heart wood
42. Cycloeucalenol C30H50O 138 Wood oil
43. 24-Methylene-cycloartanol C31H52O 122 Wood oil
44. 4,14α-Dimethyl 5α-ergosta-8,24 (28)-dien-3β-ol C30H50O-Heart wood
45. 4α-Methyl-5α-ergosta-8,24 (28)-dien-3β-ol C29H48O-Heart wood
46. Neem leaf glycoprotein—Leaf
47. Non-terpenoid and non-steroid constituents of neem (A. indica)
48. Name of constituents; Chemical formula; Melting point (° C.); Isolated from
   a. Kaemferol C15H10O6 276 Blossoms
   b. Quercetin C15H10O7 313 Blossoms and leaves
   c. Myricetin C15H10O8 357 Blossoms
   d. Sugiol C20H28O2 292 Trunk bark
   e. Nimbiol C18H24O2 250 Trunk bark
   f. Glucoside of Quercetin C21H20O12—Leaves
   g. Glucoside of Kaemferol C21H20O11—Leaves
   h. Melicitrin C20H18O12-Blossoms
   i. Quercetin-3-galactoside (Hyperin) C21H20O12 237 Blossoms and leaves
   j. Kaemferol-3-glucoside (Astragalin) C21H20O11 178 Blossoms
   k. Quercitrin C21H20O11 250 Leaves
   l. Rutin C27H30O16 214 Leaves
   m. Isorhamnetin C16H12O7 305 Leaves
   n. Rhamnoside of Quercetin C21H20O11 245 Leaves
   o. 5-Hydroxy-methyl furfural C6H6O3-Fruit

### REFERENCES

Talwar G P, Pal R, Singh O, Garg S, Taluja V, Upadhyay S N, Gopalan S, Jain V, Kaur J, Sehgal S., Safety of intrauterine administration of purified neem seed oil (Praneem Vilci) in women & effect of its co-administration with the heterospecies dimer birth control vaccine on antibody response to human chorionic gonadotropin.
Garg S, Talwar G P, Upadhyay S N. J Ethnopharmacol. 1998 April; 60(3): 235-46. Immunocontraceptive activity guided fractionation and characterization of active constituents of neem (Azadirachta indica) seed extracts. Indian J Med Res. 1995 August; 102:66-70
Juneja S C, Pfeifer T, Williams R S, Chegini N.; Neem oil inhibits two-cell embryo development and trophectoderm attachment and proliferation in vitro. J Assist Reprod Genet. 1994 September; 11(8): 419-27.

What is claimed is:

1. A method of treating cancer, the method comprising administering a compound to a subject in need thereof, wherein the compound is an anti-hCG compound, wherein the compound is an anti-hCG compound in one or more of Azadirachta indica (neem), Gossypium herbaceum, Trichosanthes kirilowii, Hibiscus rosa-sinensis (Malvaceae), Momordica charantia or Scutellaria barbata, and wherein the compound is administered in an amount that is adequate to treat cancer.

2. The method of claim 1, wherein the method comprises identifying the subject, wherein the subject is a mammal.

3. The method of claim 1, wherein the anti-hCG compound is isolated from a plant or produced synthetically.

4. The method of claim 1, wherein the cancer comprises a tumor.

5. The method of claim 4, wherein the cancer expresses a biomarker for a chorionic antigen.

6. The method of claim 1, wherein the method of treating is preventative.

7. The method of claim 1, wherein the method of treating is curative.

8. The method of claim 1, wherein the cancer is one or more of breast, liver, prostate, colon, bone, lung, kidney, uterus, or lymphocytes.

9. The method of claim 1, wherein the anti-hCG compound is a component in Azadirachta indica (neem).

10. The method of claim 9, wherein an extract of Azadirachta indica (neem) is prepared by a Soxhlet extraction method.

11. The method of claim 9, wherein the anti-hCG compound is IRDNA.

12. The method of claim 9, wherein the anti-hCG compound is IRDNB.

13. The method of claim 1, wherein the anti-hCG compound is a component in Gossypium herbaceum.

14. The method of claim 1, wherein the anti-hCG compound is a component in Trichosanthes kirilowii.

15. The method of claim 14, wherein the anti-hCG compound is trichosanthin.

16. The method of claim 1, wherein the anti-hCG compound is a component in Hibiscus rosa-sinensis (Malvaceae).

17. The method of claim 1, wherein the anti-hCG compound is a component in Scutellaria barbata.

18. The method of claim 17, wherein a proliferative effect of hCG on leiomyoma cells and myometrial cells is reduced.

19. The method of claim 1, wherein the anti-hCG compound is administered as a composition.

20. The method of claim 19, wherein the composition comprises an active form of the anti-hCG compound and an inert pharmaceutically acceptable carrier.

21. The method of claim 19, wherein the composition is formulated in a dosage form appropriate for a route of administration, wherein the route of administration is one or more of oral, parenteral (intramuscular, intraperitoneal, intravenous or subcutaneous injection), nasal, vaginal, rectal or sublingual.

22. The method of claim 19, wherein the composition is a solid dosage form for oral administration.

23. The method of claim 22, wherein the solid dosage is one or more of a capsule, a tablet, a pill, a powder or a granule, comprising an active form of the anti-hCG compound admixed with at least one inert pharmaceutically acceptable carrier.

24. The method of claim 23, wherein the at least one inert pharmaceutically acceptable carrier is one or more of sucrose, lactose or starch.

25. The method of claim 22, wherein when the solid dosage form is a capsule, a tablet or a pill, the solid dosage form additionally comprises a buffering agent.

26. The method of claim 22, wherein when the solid dosage form is a tablet or a pill, solid dosage form additionally comprises an enteric coating.

27. The method of claim 19, wherein the composition is a liquid dosage form for oral administration, wherein the liquid dosage form is a pharmaceutically acceptable emulsion, a solution, a suspension, a syrup or an elixir.

28. The method of claim 27, wherein the liquid dosage form comprises an inert diluent.

29. The method of claim 27, wherein the liquid dosage form additionally comprises an adjuvant, wherein the adjuvant is one or more of a wetting agent, an emulsifying agent or a suspending agent.

30. The method of claim 27, wherein the liquid dosage form additionally comprises one or more of a sweetening agent, a flavoring agent or a perfuming agent.

31. The method of claim 19, wherein the composition is a parenteral dosage form.

32. The method of claim 31, wherein the parenteral dosage form is a sterile aqueous solution, a sterile non-aqueous solution, a suspension or an emulsion.

33. The method of claim 32, wherein the sterile non-aqueous solution comprises a non-aqueous solvent or vehicle.

34. The method of claim 33, wherein the non-aqueous solvent or vehicle is propylene glycol, polyethylene glycol, a vegetable oil, gelatin or an injectable organic ester.

35. The method of claim 34, wherein the vegetable oil is olive oil or corn oil, and the injectable organic ester is ethyl oleate.

36. The method of claim 31, wherein the parenteral dosage form additionally comprises an adjuvant, wherein the adjuvant comprises one or more of a preserving agent, a wetting agent, an emulsifying agent or a dispersing agent.

37. The method of claim 31, wherein the parenteral dosage form is sterilized by one or more of filtration through a bacteria retaining filter, incorporating one or more sterilizing agents, irradiation or heating.

38. The method of claim 31, wherein the parenteral dosage form is prepared immediately before administration using sterile water, a sterile injectable medium or a combination thereof.

39. The method of claim 19, wherein the composition comprises an excipient, wherein the excipient is a cocoa butter or a suppository wax.

40. The method of claim 1, wherein the dosage amount is about 0.001 mg/kg to about 10 mg/kg of body weight per day.

41. The method of claim 1, wherein the method additionally comprises one or more treatment supplements selected from multivitamins, immune supplement, and green shakes.

42. A method of treating cancer, the method comprising administering an herbal substance to boost immune system in a subject, wherein the herbal substance comprises an anti-hCG compound in one or more of Azadirachta indica (neem), Gossypium herbaceum, Trichosanthes kirilowii, Hibiscus rosa-sinensis (Malvaceae), Momordica charantia or Scutellaria barbata, wherein the anti-hCG compound is administered in an amount that is adequate to treat cancer.

43. A composition for treating cancer, wherein the composition is an herbal substance to boost immune system in a subject, wherein the herbal substance comprises an anti-hCG compound in one or more of Azadirachta indica (neem), Gossypium herbaceum, Trichosanthes kirilowii, Hibiscus rosa-sinensis (Malvaceae), Momordica charantia or Scutellaria barbata, and wherein the anti-hCG compound is administered in an amount that is adequate to treat cancer.
